(19) United States
(12) Patent Application Publication
(10) Pub. No.: US 2020/0196962 A1
(43) Pub. Date: Jun. 25, 2020

(54) SYSTEMS, METHODS, AND APPARATUS FOR PERSONAL AND GROUP VITAL SIGNS CURVES

(71) Applicant: Scanadu Incorporated, Sunnyvale, CA (US)

(72) Inventors: Wenyi Zhao, Mountain View, CA (US); Brandon Dennis Woolsey, San Jose, CA (US); Walter De Brouwer, Los Altos, CA (US); Eron Anthony Villarreal, San Jose, CA (US); Whitney Morgan McGowan, San Jose, CA (US)

(21) Appl. No.: 16/231,591

(22) Filed: Dec. 23, 2018

(51) Int. Cl. A61B 5/00; G16H 40/63

(52) U.S. Cl. CPC A61B 5/743; G16H 40/63; A61B 5/7425

(57) ABSTRACT

In one embodiment of the invention, a system for periodically and simultaneously scanning for a plurality of vital signs of a user is disclosed. The system includes a personal portable wireless vital signs scanner having a pair of electrodes to form a circuit with a user's body; and a personal wireless multifunction device wirelessly in communication with the personal portable wireless vital signs scanner. Users may tag their vital signs scans with additional information. Vital signs data may be stored on non-volatile memory on the multifunction device and periodically uploaded to a vital signs storage server. The accumulated vital signs data is used to build the user's canonical curve. In another embodiment of the invention, multiple users may upload their vital signs to a group vital signs server. Group vital sign reference curves can made with the group data obtained. Group data may be compared with the user's personal vital sign data and health recommendations made accordingly to the user.

### CROSS-REFERENCE TO RELATED APPLICATIONS

This non-provisional United States (U.S.) patent application is a continuation application and claims the benefit of U.S. Non-Provisional patent application Ser. No. 14/516,575 entitled SYSTEMS, METHODS, AND APPARATUS FOR PERSONAL AND GROUP VITAL SIGNS CURVES filed on Oct. 16, 2016 by inventor Wenyi Zhao et al., pending. U.S. Non-Provisional patent application Ser. No. 14/516,575 claims the benefit of U.S. Provisional Patent Application No. 61/961,535 entitled SYSTEMS, METHODS, AND APPARATUS FOR PERSONAL AND GROUP VITAL SIGNS CURVES filed on Oct. 16, 2013 by inventor Wenyi Zhao et al.; and is a continuation in part and claims the benefit of U.S. patent application Ser. No. 14/292,820 entitled METHODS OF DATA ACQUISITION QUALITY AND DATA FUSION FOR PERSONAL PORTABLE WIRELESS VITAL SIGNS SCANNER filed on May 30, 2014 inventors Wenyi Zhao et al., including the incorporation by reference of FIGS. 5A-5E. U.S. patent application Ser. No. 14/292,820 claims the benefit of U.S. Provisional Patent Application Nos. 61/875,681 filed on Sep. 9, 2013 and 61/924,230 filed on Jan. 6, 2014.

### FIELD OF THE INVENTION

This invention generally relates to vital signs scanning by a portable device with multiple integrated sensors.

### BACKGROUND OF THE INVENTION

Healthcare is a key element of any modern society. Over the years, it has brought people the benefit of the latest technological breakthroughs that are safeguarded by well-established regulatory process. The practice of medical practitioners has also evolved into highly specialized fields and subfields. One of the most important aspects of medicine is preventive care. A significant portion of healthcare costs could be reduced if ailments are diagnosed early. Yet many of the tools to diagnose early symptoms are unavailable to the average consumer.

Vital signs of one's body, such as temperature for example, form the base map of ones health. Fluctuations in our vital signs may be predictive of undiagnosed ailments. It's important to have easy access to their vital signs as frequently as needed. Yet the average consumer has no easy method of obtaining many of their vital signs without visiting a hospital or clinic. One of the easiest-to-measure vital signs is body temperature. Consumers are able to measure body temperature at home with an inexpensive home thermometer. However the average consumer still does not have easy access to devices for measuring the other important vital signs of ones body, such as blood oxygenation or blood pressure for example. The technology is available to measure the important vital signs, but typically limited to clinics and hospitals.

Consumers do not have a way to measure all of their important vital signs at home. Consumers cannot visit their physician five or more times a day to constantly monitor their vital signs. This has put the average consumer with a medical condition into a difficult situation, where they do not know what to do with their condition when they need vital signs information while at home or traveling. The few options the average consumer has with an unknown medical condition include staying calm and doing nothing, calling their primary care providers (PCP) to get an appointment, or visiting an emergency room (ER) and waiting for hours.

Even if the consumer opted to do one of the latter options, the PCP or ER may not be able to provide personalized advice without knowing the specifics about their patients. The physician may have some idea about one's health condition based on an annual exam but the data may be outdated and useless with a current medical condition.

As a result, the average consumer may not receive the best medical care due to the lack of information. And together, with PCPs, we also manage to add more cost to the healthcare system that is already very expensive as people live longer.

The problem, simply put, is that consumer access to basic health care is rather limited. It is desirable to improve the quality and access to basic health care for average consumers.

### SUMMARY OF THE INVENTION

The embodiments of the invention are best summarized by the claims below. Insofar as a summary is required, one embodiment of the invention can be described as a portable vital signs scanner with multiple integrated vital signs sensors.

This summary is provided to efficiently present the general concept of the invention and should not be interpreted as limiting the scope of the claims.

### BRIEF DESCRIPTION OF THE DRAWINGS

These and other features, aspects, and advantages of the present disclosure will now be described with reference to the drawings of embodiments, which embodiments are intended to illustrate and not to limit the disclosure, as are described in varying degrees of detail below.

FIG. 1A is a diagram illustrating an exemplary vital signs scanning system with the scanner held at the forehead.

FIG. 1B is a perspective view of a user squeezing the exemplary vital signs scanner.

FIG. 1C is a diagram illustrating a portable wireless multifunctional device with a scan screen of vital signs scanning user interface.

FIG. 1D is another diagram illustrating an exemplary vital signs scanning system with the scanner held at a chest position.

FIGS. 1E-1F are diagrams illustrating how microphones of the exemplary vital signs scanner can capture body sounds, such as from a user's heart or lung.

FIG. 1G is another diagram illustrating an exemplary vital signs scanning system with the scanner held in fingers of each hand.

FIG. 2A illustrates an exemplary portable wireless multifunction device to execute the vital signs scanning application.

FIG. 2B illustrates a schematic representation of the components of the portable wireless multifunctional device.

FIG. 3A is an exemplary health status window displayed on the portable wireless multifunctional device by the vital signs scanning user interface (VSUI).

FIG. 3B is an exemplary scan results window displayed on the portable wireless multifunctional device by the vital signs scanning user interface.

FIG. 3C illustrates exemplary slide windows generated on the portable wireless multifunctional device by the vital signs scanning user interface.

FIG. 3D illustrates an exemplary second scan selection window of the vital signs scanning application on the portable wireless device.

FIGS. 4A-4B illustrate a temperature averaging window generated in a touch screen of the portable wireless multifunction device by the vital signs scanning software application.

FIGS. 5A-5E illustrate prognosis windows for vital signs in a touch screen of the portable wireless multifunction device.

FIGS. 6A-6B are perspective views of an embodiment of the invention.

FIGS. 6C-6D are perspective views of another embodiment of the invention.

FIG. 7A is an exploded view of the exemplary portable wireless vital signs scanner.

FIG. 7B illustrates a partially assembled exemplary portable wireless vital signs scanner

FIG. 8A illustrates a functional block diagram of electronic circuitry within the exemplary portable wireless vital signs scanner.

FIG. 8B illustrates a main printed circuit board coupled to a daughter printed circuit board with various electronic circuitry within the exemplary portable wireless vital signs scanner mounted to each.

FIG. 9 illustrates an exemplary hierarchy of the vita signs graphical user interface provided by the vital signs scanning software application executed by the personal wireless multifunction device.

FIG. 10 is a block diagram illustrating an exemplary vital signs cloud system.

FIG. 11A illustrates a basic daily vital sign curve over one twenty-four hour period.

FIG. 11B illustrates a canonical average vital sign curve including a graphical representation of standard deviations.

FIG. 11C illustrates a vital sign curve of a vital signs group plotted against a user's vital sign curve shown in FIG. 11A over a twenty four hour period.

FIG. 11D illustrates a group canonical vital sign curve of a vital signs group plotted against the user's canonical average vital sign curve shown in FIG. 11B.

FIG. 12A, illustrates an exemplary tagging screen displayed on a multifunction device.

FIG. 12B, illustrates an exemplary tagging screen with virtual keyboard to enter the tagged information.

FIG. 13 illustrates a flow diagram for giving users recommendations during the vital sign scanning process.

FIG. 14 illustrates a block diagram of an exemplary cloud server system with multiple users accessing a group server.

FIG. 15 illustrates an exemplary user acknowledgment screen of the software application.

FIG. 16 is a functional diagram of the cloud based server system of an exemplary embodiment of the invention.

FIG. 17 illustrates an exemplary method of organizing the user's data within a cloud system.

FIG. 18 illustrates a diagram of a global group of users.

FIG. 19 is an exemplary graph of the user's vital signs.

FIGS. 20 and 21 illustrate automated methods of obtaining a user's height and weight.

### DETAILED DESCRIPTION

Many alternative embodiments of the present aspects may be appropriate and are contemplated, including as described in these detailed embodiments, though also including alternatives that may not be expressly shown or described herein but as obvious variants or obviously contemplated according to one of ordinary skill based on reviewing the totality of this disclosure in combination with other available information. For example, it is contemplated that features shown and described with respect to one or more particular embodiments may also be included in combination with another embodiment even though not expressly shown and described in that specific combination.

For purpose of efficiency, reference numbers may be repeated between the figures where they are intended to represent similar features between otherwise varied embodiments, though those features may also incorporate certain differences between embodiments if and to the extent specified as such or otherwise apparent to one of ordinary skill (such as differences clearly shown between them in the respective figures).

It is desirable for consumers to take greater control of their own basic health and work with their primary care providers (PCPs) to provide personalized healthcare. Some embodiments of the invention provide a consumer device that is small enough to be carried in a pocket or purse with which effortless vital signs scans can be performed, anytime, anywhere. The consumer device, referred to as a vital signs scanner, can transfer the vital signs results to a portable wireless multifunction device, such as a smartphone, for storage and display to a user over time to illustrate health trends. The vital signs scanner allows consumers to take greater control of their own basic health and work with PCPs to provide personalized healthcare.

The vital signs scanner allows users to efficiently measure multiple vital signs simultaneously. Vital signs scanning with the vital signs scanner is quick and easy and very convenient in that it can simultaneously capture a plurality of vita signs data with one scanning session (one or two vital signs scans) at a given time and date. The vital signs data is transferred to a users own portable multifunction touch screen device, e.g. a smart phone. The portable multifunction device, with the assistance of vita signs scanning software, displays the scanning results in an intuitive user interface that is simple to understand.

The vital signs scanning device provides a method of vital signs scanning to help solve the missing information link so a user can take control of managing his/her own health. In addition to providing vital signs scanning, the vital signs scanner and system also stores the users vital signs measurements and trends over time of a day and date. The vital signs scanner and system provides easy access (almost anywhere at anytime) to important vital signs measurements such as blood oxygenation, blood pressure, heart rate, etc. The vital signs scanner and system can help share up-to-date vital signs data with a user's PCP for better diagnosis of medical conditions. Perhaps even more importantly, sharing of history and trends of vital signs data before and after an ailment with the user's PCP can provide clues to its cause and not just indicate the symptoms.

The personal wireless vital signs scanner combines aesthetic design with functionality. The personal wireless vital signs scanner is light weight and easily fits into one hand. The personal wireless vital signs scanner can be held and operated with just two fingers of one hand. The user's other hand is free to hold a smartphone with a vital signs scanning application running to control the vital signs scanning process and view the scanning results. Vital signs data of a users body can change at different times of each day. The personal wireless vital signs scanner is so small, light, and esthetically pleasing that a user may desire to take it with them to perform a plurality of vital signs scans at different times throughout his/her day over a plurality of days.

A portable vital signs scanner and system may prove to be useful for healthcare professionals as well. For example, patients could scan for their own vital signs themselves in a busy hospital, clinic or doctors office, rather than wait in long lines just to get a simple checkup before seeing the doctor. The patients scans are then uploaded to a server at the hospital, clinic, or office. With these self-obtained vital signs scans of patients being uploaded to a server, medical assistants and nurses, ordinarily checking for vital signs, can better spend their time curing the ailments of the patients.

The self-obtained vital signs scans of patients may also serve to triage the patients that are waiting for medical care. For example, a self-obtained vital signs scan of a patient indicating an elevated or irregular heart rate may signal hospital staff to attend to this patient immediately or at least a higher priority in a queue of patients. In this manner, the self-obtained vital signs scans of patients provide a clinic staff with a sense of the severity of the condition of patients waiting and can make appropriate schedule priority adjustments, if needed.

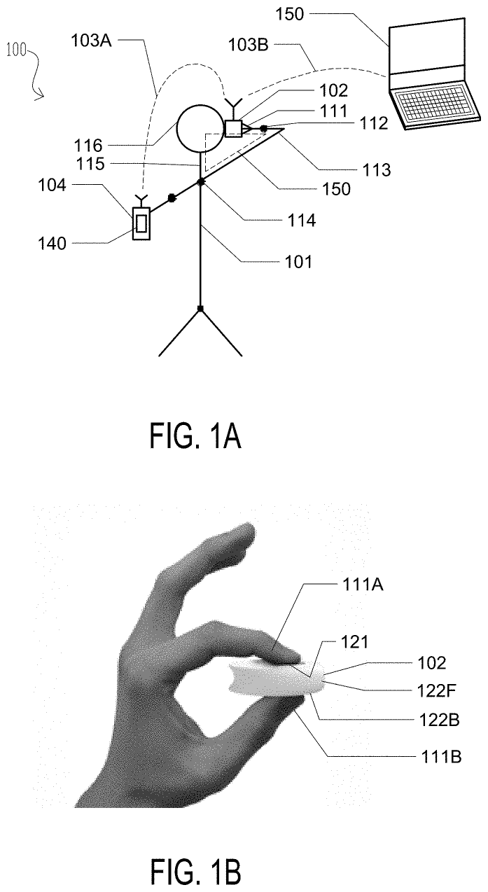

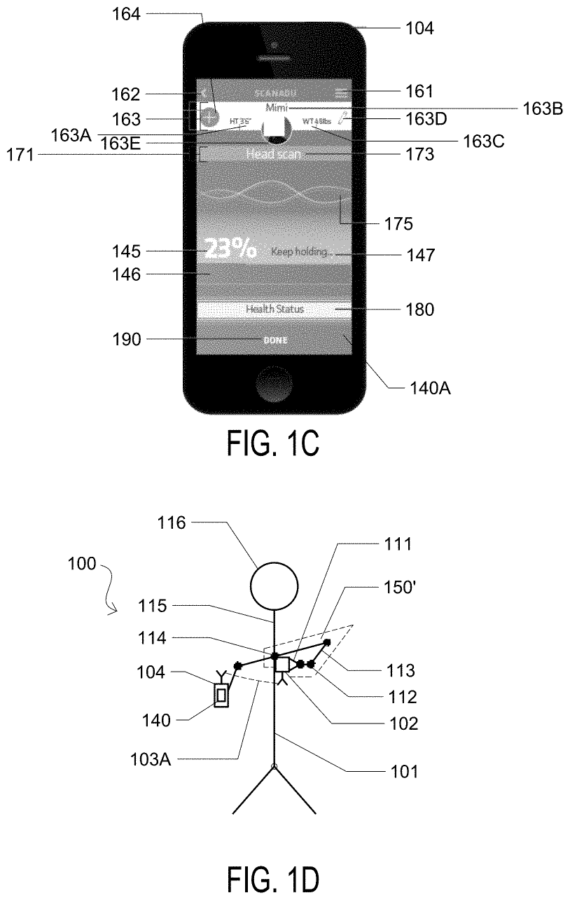

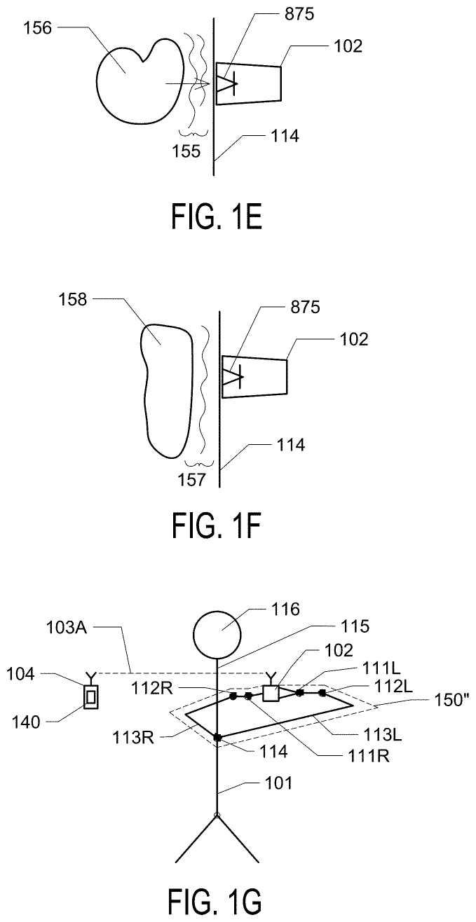

Referring now to FIG. 1A, a diagram illustrating a vital signs scanning system 100 is shown. The scanning system 100 includes a portable wireless vital signs scanner 102 and a portable wireless multifunction device 104 in wireless communication with each other over a wireless communication channel 103A. The vital signs scanner 102 includes a plurality of sensors designed to read vital signs from a user's body 101. An instance or snap shot of vital signs, such as temperature, heart rate, blood oxygenation or SpO2, ECG (electrocardiogram), and possibly stress levels, all synchronously measured, can be reported to the device 104 by the scanner 102 in less than a minute. Exemplary methods and algorithms for determining one or more of these vital signs from the sensor data are described in International Application No. PCT/US2013/061046, filed by Scanadu Corporation on 19 Oct. 2012, having international publication no. WO 2013/066642, entitled AUTOMATED PERSONAL MEDICAL DIAGNOSTIC SYSTEM, METHOD, AND ARRANGEMENT, claiming priority to U.S. Pat. No. 61/549,134 filed on 19 Oct. 2011, and is hereby incorporated by reference.

The algorithms and processes disclosed in International Application No. PCT/US2013/061046 are based upon one or more of the following references (all of which are incorporated herein in their entirety): Pulse transit time: an appraisal of potential clinical applications, Thorax 1999; 54:452-457 [doi:10.1136/thx.54.5.452][http://thorax.bmj,com/contentl54/5/452.full]; U.S. Pat. Nos. 6,723,054; 6,527,728; U.S. Publication Nos. 2007/0276632; 2003/0199771; Severinghaus, John W., Honda Yoshiyuki (April 1987), “History of Blood Gas Analysis. VII. Pulse Oximetry”, Journal of Clinical Monitoring #(2): 135-138; Millikan G. A. (1942). “The oximeter: an instrument for measuring continuously oxygen-saturation of arterial blood in man”, Rev. Sci. Instrum 13 (10): 434-44 [doi:10.1063/1.1769941]; U.S. Patent Nos. 6,385,471; 5,934,277; 5,503,148; 5,351,685; 5,259,381; 4,883,353; 4,824,242; 4,807,631; 4,796,636; 4,714,080; 4,623,248; and 4,266,554.

Integration of multiple sensors and scan quality algorithms make it possible to monitor the quality of the scanning process and then provide intuitive user feedback to control the interactive scanning process, to make a great user experience in the vital signs scanning process.

The wireless vital signs scanner 102 may perform vital signs scans and display the results in under a minute. Generally scans may be completed in approximately ten seconds. The length of a scanning session may depend on the user's ability to correctly utilize the scanner 102. For example, if the user moves too much during the scanning session, the session will last longer as the device 104 prompts the user to remain still. Different types of scans may also take different lengths of time. For example, in a standard ten second scan where the scanner is held against a user's forehead, temperature, SpO2, ECG, heart rate, blood pressure may be measured. For a 30 second extended head scan, vital signs such as blood pressure and heart rate variability (related to emotional stress) may be captured. For a thirty second scan from a user's chest, respiration rate and body sounds may be measured or collected. In any case, the scanning sessions are still short and convenient.

Short scanning sessions have several advantages. A short scanning session allows a user to take a quick break from their daily activities to perform a scan anywhere and at any time. The ease and rapidness of performing a vital signs scan will encourage users to perform the scan multiple times a day, providing more complete and accurate trending data. The invention provides a consumer oriented scanner that a user can use anytime anywhere to obtain multiple vital sign measurements in seconds.

Short scanning sessions also conserve power. With ten second scans, the scanner is designed to last for one week of normal usage with one full battery charge. If the power is on for a total of about 30 seconds for each scan, then total power-on time for each day is less than one hour with 100 scans per day. The scanner 102 may operate for a week at a time between battery recharging sessions.

Scanner 102 is an elegant consumer device that is portable. Unlike other vital sign monitors, scanner 102 does not need to be worn. Scanner 102 is perhaps the smallest consumer device that can measure multiple vital signs simultaneously. Measuring approximately 60mm in diameter and 18mm high, the scanner 102 can be easily places in a pocket or purse for use at any time convenient to the user. At any time the user has a moment to spare, the scanner 102 may be used to obtain multiple vital sign measurements by simply finger-holding it against the user's forehead.

Using a multifunction device 104 to display the vital signs scanning results allows the scanner 102 to maintain a compact size and minimalist form. Multifunction device 104 may be any portable wireless multifunction device such as a smartphone, tablet PC, or the so called smart watches. Generally these devices are pre-owned and already available to the average consumer, so utilizing the display capabilities of multifunction device 104 does not detract from the portability of the invention. The ubiquity of smartphones also means that the average consumer does not need to pay more for a dedicated display device. Combining the vital signs scanner 102 with, a smartphone that a user already has, allows one to take control and greater responsibility for his/her health without sacrificing valuable time and money.

To display the vital signs scanning results, the portable wireless digital device 104 executes a vital signs scanning software application 140. The instructions of the vital signs scanning software application 140 are executable with the operating system, (e.g., Android and iOS), of the multifunction device 104. Once the software application is active, the user may power up the vital signs scanner 102. Upon power up, the vital signs scanner 102 is paired with the portable wireless digital device 104 to form the communication channel 103A between them. Accordingly, each of the scanner 102 and multifunction device 104 has a compatible wireless radio to form a compatible wireless communication channel. In one embodiment, the communication channel 103A is a Bluetooth version 4, a smart low energy (LE) supported channel that each wireless radio supports. The vital signs scanner 102 sends the vital sign information wirelessly to the portable wireless multifunction scanner 102 over the wireless communication channel 103A for storage and further analysis.

With the communication channel 103A available, the vital signs scanner 102 is pressed against a user's forehead. The forehead is identified as the single place with enough blood vessels and thin skin so that temperature, pulse oximetry and ECG can be obtained in sync and time-stamped. A scanning button is pressed on the user interface of the application 140 of the portable wireless multifunction device 104 to start the scanner 102 scanning for vital signs information of the user. After scanning for approximately 10 seconds or less, the vital signs scanner 102 sends the vital sign information wirelessly to the portable wireless multifunction device 102. The multifunction device 104 may display the results of the scan on a touchscreen display.

The vital signs scanner 102 is used periodically to scan for vital signs each day. Statistical information regarding a plurality of scans each day over a plurality of days can be generated and displayed on the touchscreen display device of the device 140. The vital signs scanning software application 140 informs a user of how those vital sign measurements may change during times of a day and over a plurality of days.

An important aspect of the invention is the quality of the scanning results. To optimize the scanning session results, the scanner 102 is designed to be easy to use to minimize user error. Similarly, the scanning software application is intuitive and easy to use. With minimal instruction, an average user can generate medical grade vital signs scans within minutes of using the invention for the first time.

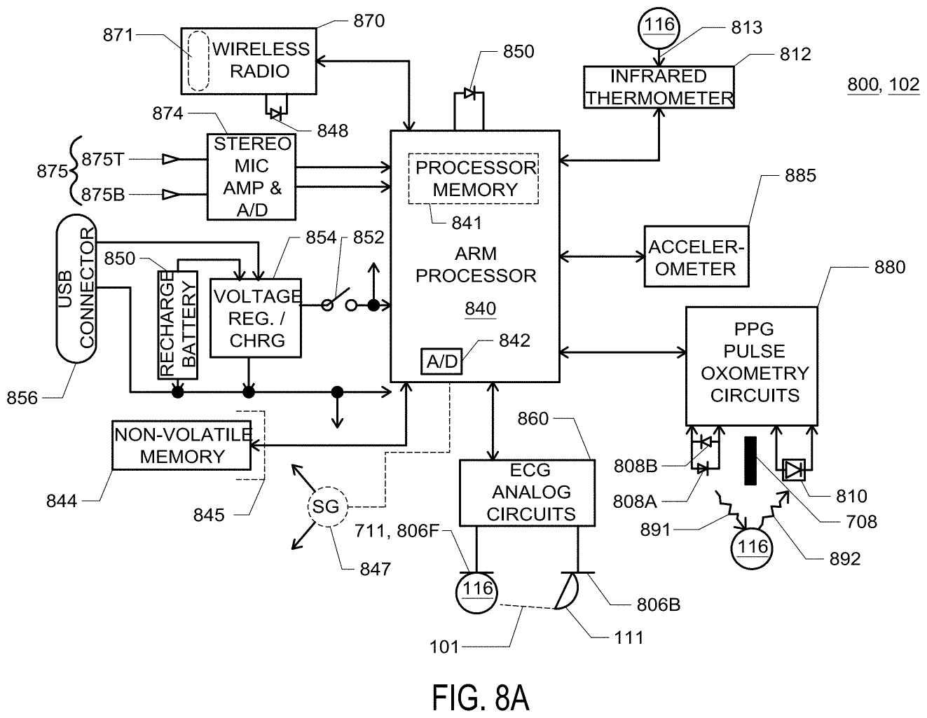

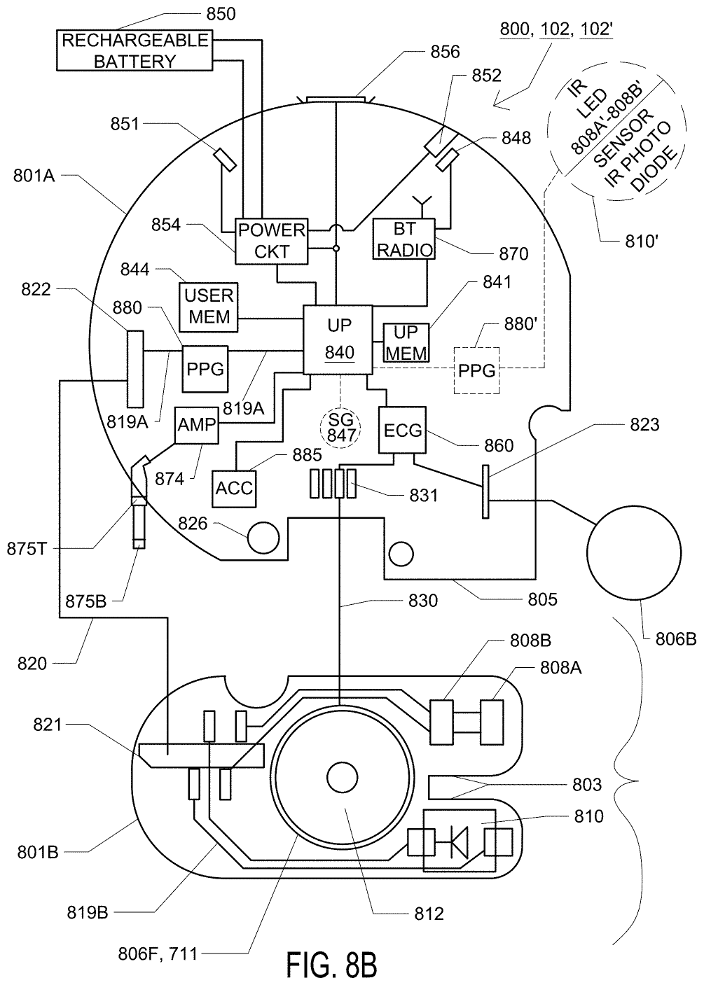

To further optimize scanning results, scan quality algorithm monitor the vita signs scanning process and provides feedback (visual and/or audible) to the user through the multifunction device 104, and/or alternatively an optional sound generator (see audible sound generator 847 in FIGS. 8A-8B) in the scanner 102. The user feedback may help the user to perform a better vital signs scan with the wireless vital signs scanner and acquire good quality vital signs measurements.

Integration of multiple sensors allows for synergistic accuracy of vital signs scans. For example, integration of an accelerometer enables motion detection that is often associated with poor signals of pulse oximetry and ECG. In another example, abnormal signals of both pulse oximetry and ECG suggest the device is not held against the body properly. This can be further confirmed by comparing the surface temperature and ambient temperature of the sensor when not in touch with the user. Quality checking of individual vital sign measurements is based on fusion of multiple sensors, including a motion sensor, such as an accelerometer. Signal quality is checked based on dynamic range detection and thresh-holding. To make the process more robust, known signal processing techniques, such as envelope detection, can be applied to the raw signals from the sensors as a preprocessing step. Quality checking of raw sensor signals from the sensors makes sensor data fusion more robust by rejecting bad signals. Thus, fusing results of multiple sensors can provide better individual measurements of each vital sign.

The intuitive scanning user interface (UI) is designed, in combination with scan quality algorithms and the device's self-diagnostic capability, to help users to finish a vital signs scan successfully. There is the quality indicator from the quality algorithm, the progress bar, and texts that provides feedback to the user to ensure a successful scanning session. For example, suggestions to “hold still” or “hold device to your forehead” may prompt the user to correct his/her poor scanning behavior.

The scanning system 100 is user friendly so that it can be used multiple times during the day to obtain data about a user's body 101. One person or one family can exclusively use the scanning system 100 and scanner 102 at home as a personal vital signs scanner. In this manner, a measure of one's personal health and medical data can be obtained right at home with the scanning system 100 without seeing a doctor or being admitted to a hospital. Each scan only lasts approximately ten to thirty seconds and obtains multiple vital sings measurements so users can take the scan repeatedly throughout the day without being inconvenienced.

The scanning system 100 can be used to personally analyze and track one's own vital signs to see various trends over time. Accordingly, the vital signs data can be accumulated over a plurality of days and a plurality of scans at various times each day, then stored in non-volatile manner with the device 104 so the data does not get lost. The vital signs data can also be backed up to a computer, a storage device, or storage server so it is not lost if the device 104 is lost or stolen. The storage server having greater storage may also be used to accumulate ones user data over a plurality of years when the device 104 is limited by its built-in storage capacity.

In operation, the vital signs scanning system 100 forms an electrical circuit 150 with the user's body 101. The circuit 150 is formed between first and second electrodes of the portable wireless vital signs scanner 102. From a first electrode of the scanner 102, the circuit 150 is made with the fingers 111, the hand 112, the arm 113, the chest 114, the neck 115, and the head 116 of the user's body 101 to a front electrode. Preferably, the portable wireless vital signs scanner 102 forms an electrical connection to the forehead portion of the head 116 of a user's body 101. Fingers 111 not only serve to hold the scanner 102, but also as one contact point for one-lead ECG (the other one-lead ECG contact point is forehead). Preferably the thumb finger 111 in one embodiment and the index finger in another embodiment forms an electrical connection with the portable wireless vital signs scanner 102.

The vital signs scanning system 100 may optionally include a personal computer 150 in wireless communication with the portable wireless vital signs scanner 102 over an alternate or additional wireless communication channel 103B.

Referring now to FIG. 1B, a perspective view of a user's fingers 111A-111B squeezing the vital signs scanner 102 is shown. The vital signs scanner 102 is squeezed between the user's fingers to form at least one electrical connection. The front side sensors and a front electrode in the vital signs scanner 102 are then pressed against the user's forehead to form an addition electrical connection. The small size 60 mm×60 mm×18 mm allows the scanner 102 to be held by just two fingers of one hand. At a weight of approximately 60g, the scanner 102 may be used by just about any person, from a child to the very elderly. Finger-held form-factor, ten to thirty seconds per scan, scan quality algorithm with feedback and an intuitive scanning user interface on a personal portable multifunction device, all help make vital signs scanning fast and easy while producing quality results.

Preferably, the scanner 102 is held between the thumb 111B and forefinger 111A of the user's left hand. The forefinger 111A may also rest over a sensor 121 and forms an electrical connection to an electrode around the sensor in one embodiment. In another embodiment, the thumb finger 111B makes contact with a bottom electrode 122B. The thumb of the left hand couples to the bottom electrical contact (electrode) on the bottom-housing portion of the scanner.

The forefinger makes contact with a rectangular glass plate over an oximeter sensor 121 in one embodiment. In another embodiment, the oximeter sensor 121 is moved to the front side of the vital signs scanner 102 so that extraneous light is less likely to interfere with the its readings.

A front side electrode 122F makes contact with the user's forehead or temple, when it is pressed up against his/her head. An infrared (IR) thermometer sensor is combined with the front side electrode 122F. The IR thermometer sensor makes temperature readings at the user's forehead. An oximeter sensor may also be located near the front side electrode 122F.

With the thumb finger 111B in contact with the bottom electrode 122B, a circuit may be formed through the finger and the hand of the user and a portion of his body back to the front side electrode 122F in the vital signs scanner 102. Once proper placement of scanner 102 is made, a scan button is selected in the software application 140 of the device 104 to command the scanner to scan the vital signs from the user's body and forward them to multifunction device 104.

Referring now to FIG. 1C, an exemplary initial scan screen window 140A of the vital signs scanning application 140 is illustrated. The initial window 140A includes an instruction scan messaging 147 with instruction scan message text and optionally an instruction figure to show the user how the vital signs scanner 102 is utilized. As indicated by the instruction scan message, the user is to keep holding the scanner to the user's left temple for the best scan.

The initial window 140A further includes a menu button 161, a back button 162, an edit button 163D, a tag information button 164, a health status button 180, and a done button 190. The tag information button 164 is used to add user information as well as tag scans with the circumstances under which a scan was undertaken, such as after eating or after exercise. The initial window 140A includes a user information bar 163 including information regarding a user's height, user's name 163B, user's weight 163C, and a user's profile picture 163E. In this manner, the user is clear as to whom is logged into the vital signs scanner user interface and for who's body is to be canned. The initial window 140A further includes a scan type indicator 173, indicating a head scan type 173A or a chest scan type 173B. The initial window 140A further includes a scan quality indicator 175, a scan progress bar 146, and a scan progress percentage indicator 146. The scan quality indicator is one form of quality feedback that may be employed by the scanning system to inform and train the user to acquire better scan data. The initial window 140A further includes a scant type slider 171 to select the type of scan that is to be performed. The menu button 161 can take the user to the next screen or a different screen within the vital signs scanning user interface. The edit button 163D can edit information and select options that are available in the vital signs scanning application 140.

The vital signs scanning application 140 may include an option to enter the user's symptoms by selecting the health status button 180. A photo may also be taken of the medical condition of a user by use of a camera in the device 104 and a photo button. Additionally, a user may add a note to his health status using the device 104 and an add note button.

The status of the scanner 102, such as powered on/off, blue tooth connection, battery charge status, and/or ready to scan, may also be displayed in one or more of the user interface windows.

The scanner 102 can collect a diverse set of physiological information (e.g., vital signs) during one or two acquisition periods totaling approximately sixty seconds (head scan, extended head scan, and/or chest scan).

Referring now to FIG. 1D, a diagram of an exemplary vital signs scanning system with the scanner held at the chest position is illustrated. In this embodiment, vital signs are first acquired from a first 10-second scan at the forehead as shown in FIG. 1A. Vital signs may the further be acquired by secondary scans such as a longer or extended scan at the forehead as shown in FIG. 1A, and then a subsequent scan conducted near the chest of the user as shown in FIG. 1D.

A secondary extended scan at the forehead may be over a range of time from about thirty seconds up to a minute so that measures of heart rate variability and respiration rate may be obtained. The secondary extend scan at the forehead can also provide for a more robust and accurate measurement of blood pressure. In terms of using the scanner, the primary and secondary scans at the forehead may occur in one single scan (e.g., 10-second or 30-second) or two separate scans (e.g., a first at 10 seconds and then a second at 30 seconds).

The secondary extended scan near the chest, a chest scan, is mainly to capture vital signs of respiration rate and additional physiological information from the captured body sounds. The vital signs scanned at the chest area may also include heart rate variability. The secondary extended scan near the chest may last for a period from thirty seconds to a minute. The vital signs scanning application executed on the multifunction device 104 may prompt the user for one or both scan locations.

The secondary chest scan can be selected by the scan type slider 171 shown in FIG. 1C. A second or third scan may be selected with a finger swipe to perform the second scan or the third scan at the chest of the user. If only the first head scan was desired, a done button 190 may be selected to avoid the secondary scans. This may be because its inconvenient due to timing or to perform against ones chest with the vital signs scanner, such as when it is inconvenient to do so in public.

As mentioned herein, a chest scan may be performed with the scanner 102 as shown by FIGS. 1D, 1E, and 1F, for example. In FIG. 1D, a second circuit 150′ may be formed with the users body 101 between the electrodes of the scanner 102. The second circuit 150′ in this case includes the chest 114, the arm 113, the hand 112, and the finger 111 of the user.

In an alternate embodiment, another circuit 150″ may be formed with the users body between the electrodes of the scanner 102 while the device 104 is nearby. This alternate circuit 150″ is formed by fingers on different hands coupling to the electrodes of the scanner 102. A left finger 111L may couple to a bottom or top electrode in the scanner 102. A right finger 111R may be coupled to the front electrode of the scanner 102. From a left finger 111L in a left hand 112L of the user, the circuit in the body includes, the left finger 111L, the left hand 112L, the left arm 113L, the chest 114, the right arm 113R, the right hand 112R, and a right finger 111R, such as shown in FIG. 1G, to complete a circuit with the scanner 102.

In either case, the ECG circuitry in the scanner 102 may then obtain further data regarding heart activity of the user that can be combined/fused with the heart activity data of a first scan, to improve the measure of vital signs of heart activity. The vital sign measures of heart activity may then be sent to the device 104 for display to the user on its built-in touchscreen display.

Temperature of the body adjacent the user's chest 114, if reliable, may also be used by the scanner to improve scanning results of temperature. Temperature at the user's finger 111R, if reliable, may also be used by the scanner to improve scanning results of temperature.

With the scanner against the users chest, an accelerometer (see accelerometer 885 in FIGS. 8A-8B) in the scanner 102 may be used to capture movement of the chest as a measure of respiration rate. The vital signs data from these measures are computed by the processor 840 and then sent to the device 140.

FIGS. 1E and 1F illustrate the use of the microphones in the scanner 102 to capture body sounds around the chest, such as heart sounds and lung or breathing sounds. These body sounds may be recorded to capture another symptom of a user's medical condition. Body sounds that are captured may also be used to judge the quality of the vital signs scanning process. The recorded body sounds may be stored locally in the memory of the scanner and/or sent to the device 140 for storage with the vital signs data of the same time and date.

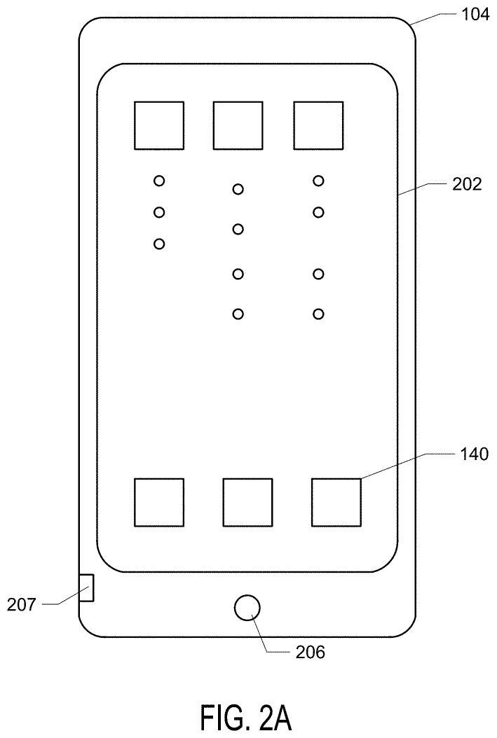

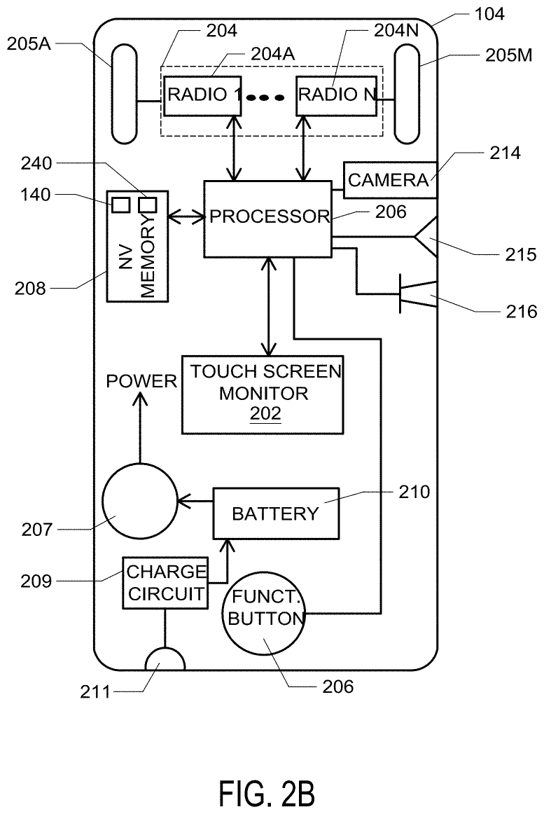

Referring now to FIG. 2A, a portable wireless multifunction device 104 is illustrated that can execute the vital signs scanning application 140. The portable wireless multifunction device 104 includes a text screen 202, at least one function button 206, and a power button or switch 207. The multifunction device 104 may display a plurality of application icons on the touch screen 202. One of these icons may be the vital signs scanning application software icon 140.

Referring now to FIG. 2B, a block diagram of the personal wireless digital device 104 is illustrated. The portable wireless multifunction device 104 may be a smart phone, a tablet computer, a portable music player, or a wireless portable storage device, for example, that include a processor, a touch screen, and a memory from which application software instructions may be executed.

As shown in FIG. 2B, the portable wireless multifunction device 104 includes a touch screen monitor 202, one or more wireless radio transmitters-receivers (wireless radios) 204A-204M coupled to their respective antenna 205A-205M, a processor 206, non-volatile memory 208, at least one function button 206, and a cover button 207 that can switch power on to each electronic circuit within the portable wireless multifunction device 104. At least one of the wireless radios 204A-204M are compatible with the wireless radio in the wireless vital signs scanner 102.

The portable wireless multifunction device 104 may further include a camera 214, a microphone 215, and a speaker 216 coupled to the processor 206 as shown. Furthermore the portable wireless digital device includes a battery 210 coupled to the power button 207. Typically the battery 210 is a rechargeable battery such that an external power source may be coupled thereto via an external power connector 211 and a charge circuit 209.

Non-volatile memory 208 of the personal wireless digital device may store the vital signs scanning application software 140 and data 220 related to the vital signs scan application software. The processor 206 can read and write to the non-volatile memory such that the vital signs scanning application software can provide a user interface to a user via the touch screen display device 202. As discussed previously, the initial vital sign scanning window 1401 may be provided as shown in FIG. 1C.

The camera 214 of the portable wireless digital device 104 may take photographs of a user's conditions or symptoms via the photograph entry button 175 of the user interface. The photographs may be stored as part of the data 240 in the non-volatile memory. The microphone 215 in the portable wireless multifunction device 104 may optionally be used to capture body sounds similar to the microphones in the scanner 102, as is shown in FIGS. 1E-1F.

The speaker 216 of the portable wireless digital device 104 may optionally be used to provide audible user feedback to the user of the vital signs scanner 102 to improve the vital signs scan quality as is discussed herein.

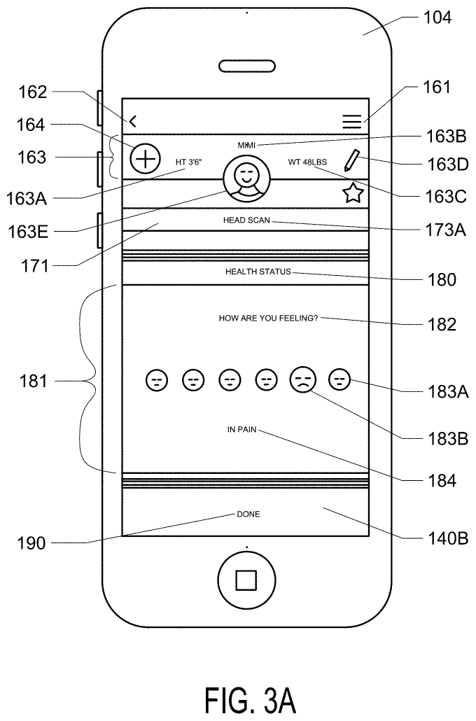

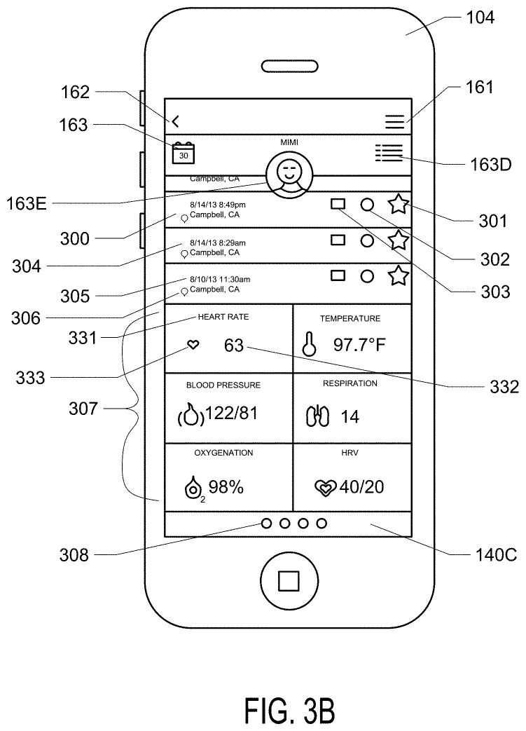

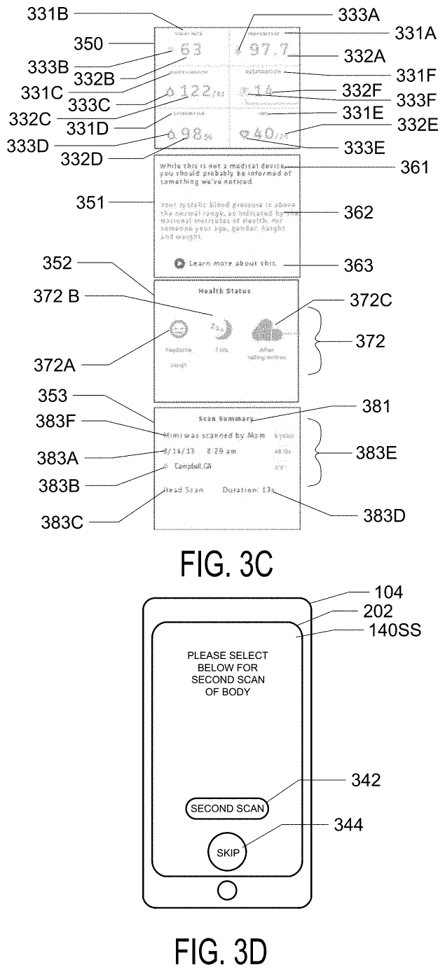

Referring now to FIG. 3A, an exemplary scanning window 140A is shown being displayed by the touch screen display device 202 of the portable wireless multi-function device 104. The scanning application software 140 generates the various images consisting of a scanning progress bar 310, a scanning icon 312, a first vital signs graph 314A, a second vital signs graph 314B, one or more result buttons 320, and one or more status icons 324.

The status icon 324 may be a wireless connection status icon indicating that the portable wireless digital device 104 is connected to the vital signs scanner 102. The button 320 may be a results button to which to switch to another scanning window/screen of a user interface provided by the scanning application software 140. The scanning icon 312 may include the plurality of color bars 312A-312E that randomly vary in color and length to indicate that scanning is occurring. The scanning progress bar 310 illustrates the progress of the scanning session being performed by the portable wireless vital signs scanner 102. In this case data is being sent from the scanner 102 to the portable wireless multifunction device 104.

Briefly referring back to FIG. 1C, the initial window 140A includes a user menu button 161 that may be used to display a users menu on how to operate the vital signs scanner. The initial window 140A may further be changed to a graph window to show plots of prior scan data stored in the device 104. A graph button may be provided to do so or a finger swipe may be used.

The first vital signs can be displayed in graph form over different day granularity such as 1 day, 1 week, 1 month, 3 months, 9 months and 1 year, such as those illustrated in the attached appendix. Graphs may be used to illustrate show the user's heart rate or heart rate variability. The waveforms displayed in the graphs are captured by the scanning process of the portable wireless vital signs scanner 102. A second vital signs graph may be oxygenation graph related to photoelectric plethysmogram (PPG) from the data obtained by the pulse oximeter. The scanner captures a user's blood volume pulse of both oxygenated and deoxygenated blood. From the photoplesmography waveforms (oxygenated and deoxygenated) a user's oxygen saturation can be obtained and displayed in the oxygenation graph.

Referring now to FIG. 3A, a health status window/screen 140B of the user interface software 140 is shown being displayed by the touch screen display device 202 of the device 104. The screen 140B includes a number of similar items illustrated in screen 140A of FIG. 1B and are not repeated here. The health status slider window/screen 140B (it can be slid sideways) includes a health status button 180, a health status window 181. The health status slider window 181 includes a display of a health status question 182 to obtain further information from a user. To respond, the health status slider window 181 includes a plurality of selectable health status response indicators 183A. The user selects one which becomes highlighted over the others, such as health status response selected icon 183B. The health status slider window 181 further includes a health status selected response information 184, such as “In Pain”, that is displayed to the user to confirm the selected health status response.

Referring now to FIGS. 3B, a scanning results window/screen 140C is shown being displayed by the touch screen display device 202 of the device 104. The screen 140B includes a number of similar items illustrated in screen 140A of FIG. 1C and are not repeated here. The exemplary scanning results window 140C may be generated by the user after selecting a results button under the menu button or by one or more sliding finger gestures (e.g., down and to the right).

The scanning results window 140C includes a results filter button 163D, and a calendar button 163. The scanning results window 140C displays one or more scanning sessions 300 each including a completed scan type indicator 301, an interpretive message indicator 302, a picture tag indicator 303, a date/time stamp 305, and a location time stamp 306. The scanning session that is selected for display on the device 104 is highlighted by a selected scan indicator 304. The scanning results window 140C further displays a results information slider window 307 and slider number indicators 308.

Referring now to FIGS. 3B-3C, the results information slider window 307 can be slid sideways by a users finger to display different slides. As shown in FIG. 3C, the different slides include a vital sign measurements results slide 310, an interpretive message slide 311, a health status report slide 312, and a scan summary information slide 313.

The vital sign measurements results slide 310 includes a plurality of vital signs icons 333, a plurality of associated vital measurements 332), and a plurality of associated vital measured labels 331. The vital measured labels 331 indicated may be heart rate, breathing rate, temperature, blood pressure, and oxygenation. The associated vital signs icons 333 may be a heart icon, a breathing icon, a thermometer icon, a blood pressure icon and an oxygenation icon respectively.

The actual measurements captured during the scanning process are illustrated by the numeric number values of the vital sign measurements 332. For example, the heart rate of 63 is shown near the heart icon and the heart rate text. The numeric values of the vital measurements 332 may be the average measurements captured during the scan that was immediately performed recently or that scan session is selected by the user. The measurements 332 are illustrated near their respective icons 333 and the respective text label 331 indicating the vital sign that was measured. The results of the scan are typically automatically saved. However, a function button may be required to delete those scan results from the wireless portable multi-function device 104 or alternately a button to upload those results to a storage server.

The interpretive message slide 311 includes a medical information disclaimer 311A, a medical interpretive message 311B, and a learn more link 311C. The medical disclaimer message slide 311A may include a message such as “while this is not a medical device, you should probably be informed of something we've noticed”. The medical interpreter message slide 311B may be something such as “your systolic blood pressure is above the normal range, as indicated by the National Institutes of Health, for someone your age, gender, height and weight.” The learn more link 311C may include a selectable icon or text to transfer the user to a web browser and a health link where he may learn more about his or her condition.

The health status report slide 312, includes health status response buttons 312 A. Health status response buttons 312A may include a headache, a cough, indication of taking medication a sleeping response button including number of hours of sleep.

The scan summary information slide 313 includes a scan summary of a selected scan session. The scan summary may include the time and date stamp, the location of the scan, the type of scan (e.g., head or chest), and the duration of the scan, such as 13 seconds. The scan summary may further indicate the age, weight, and height of the user being scanned as well as whom performed the scan. In the example illustrated, the user Mimi was scanned by her Mom.

FIG. 3D is an illustration of an exemplary window of the vital signs scanning application on the portable wireless device. In this exemplary window displayed on touch screen 202 of the multifunction device 104, the vital signs scanning application 140 is prompting the user to select a second scan. A second scan may be selected by touching scan virtual button 342 or using a finger gesture on the touch screen. A third scan may also be selected after the second by touching scan virtual button 342 or using a finger gesture on the touch screen. The third scan may be performed at the chest region to measure respiration rate and collect body sounds. The user may desire to skip a secondary scan by touching a skip scan virtual button 344.

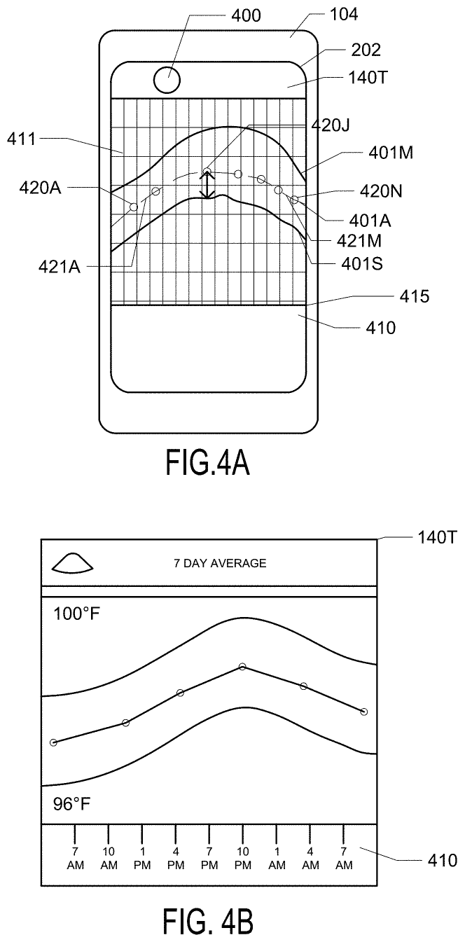

Referring now to FIGS. 4A-4B, a temperature averaging window 140C is shown being illustrated in the touch screen 202 by the scanning application software 140. This may be displayed as a result of selecting the graph button 165 of the initial scanning window 1401. The temperature averaging window 140C could include a textual heading 400 illustrating the types of graph that are plotted below. The textual heading 400 may recite “seven-day average” to let a user know that one or more seven-day average graphs are being displayed below. The portable vital signs scanner 102 may be used periodically throughout a 24-hour period each day. The seven day average may look back over a seven day window and time, plotting an average curve 401A, a maximum curve 401M, and a minimum curve 401S. The vital sign measurements are plotted on the Y-axis 411 and a time as the time of day on the X-axis 410. The portable wireless vital signs scanner 102 is expected to be used daily at multiple times during a day. In this, manner the vital signs of the user are captured periodically during the day by the vital signs scanner 102 and the personal portable wireless device 104 of the scanning system 100. The maximum curve 401M and the minimum curve 401S may be illustrating plots of the maximum value and minimum values over all scans that were previously performed. The time of day axis 410 illustrates periodic time values during the span of a 24-hour day. In one embodiment, the far most right point of the curves represents the given time of day 415 of a sliding window. In another embodiment, the time axis is fixed and the curve 401A grows from left to right during the time period as scans are made and time actually progresses. The scan points 420A-420M are illustrated along the average curve 401A. The scan points 420-420M may represent actual scans during the day or some measure of average during the preceding seven-day period. Interpolation lines 421A-421M may be inserted between each scan point to show a trend line of how the vital sign that is measured varies during times of the day. For example, scanning point 420J may represent a scan that took place between 4:00 and 7:00 pm and how the body trends towards that during that time of day.

The illustrated seven day average graph illustrated in FIG. 4 shows a body temperature graph. This is for illustration purposes only. The vital sign measurement curves could be temperature curves, blood pressure curves, oxygenation curves, heart rate curves, breathing/respiration rate curves, for example, that represent measurements that are scanned by the vital signs scanner 102.

As more information is captured by the scanner 102 and stored in the personal portable multi-function device 104, additional results may be plotted over time to generate the curves for display by an averaging window, such as vital signs window 140C.

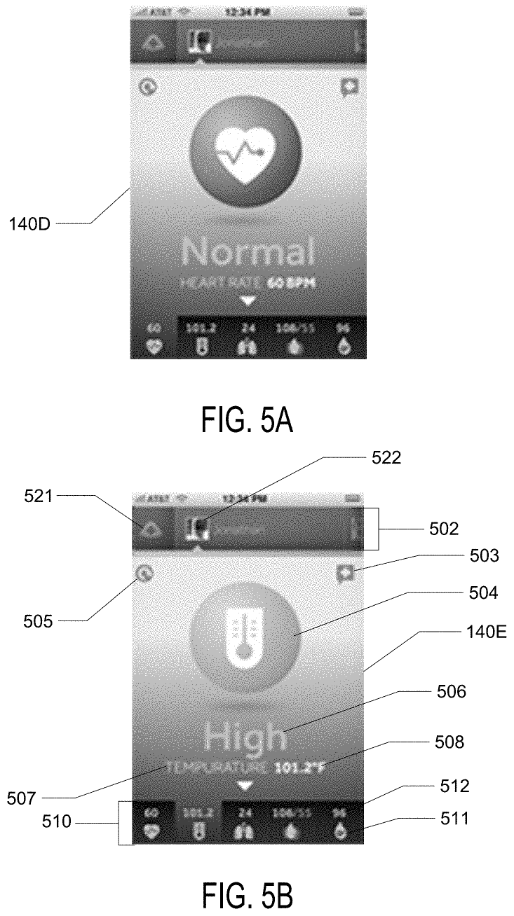

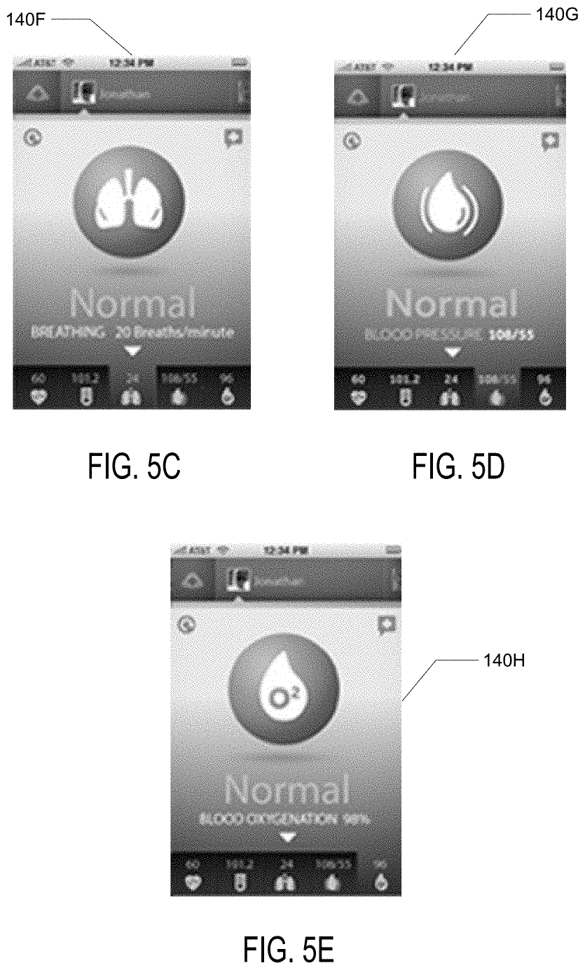

Referring now to FIG. 5A-5E, a plurality of prognosis windows 140D-140H are illustrated. In FIG. 5A, the heart rate prognosis window is shown. In FIG. 5B, the temperature prognosis window 140E is illustrated. In FIG. 5C, a breathing rate prognosis window 140F is illustrated. In FIG. 5D, a blood pressure prognosis window 140G is illustrated. In FIG. 5E, a blood oxygenation window 140H is illustrated. These windows may be selected through the use of the vital signs icons 333A-333E acting as buttons to display the respective prognosis window.

As illustrated in FIG. 5B, each prognosis screen 140D-140H, may include a navigation bar 502, one or more function buttons 503, a vital signs icon 504, a return button 505, a conditions indictor 506, a vital signs indicator 507, a measurements value indication 508, and a vital signs bar 510. The navigation bar 502 may allow a user to navigate the various screens of the vital signs application scanning software 140. For example, a scan screen icon/button 521 may be provided to jump to the scanning screen. A prognosis screen icon/button 522 may be provided to jump to the prognosis screens 140D-140H.

The vital signs bar 510 may be provided to navigate through the various vital signs prognosis windows/screens 140D-140H as well as providing a snapshot of the values of each of the vital sign measurements. In that case the vital signs bar 510 includes a measurement value indicator 512 and a vital signs icon 511 for each of the vital signs that are scanned and captured by the vital signs scanning system 100.

The return button 505 may be used to return to the previous screen that was displayed by the user interface of the scanning application software 140. The function button 503 may be an add a note button to add text about a user's condition or circumstances under which a scan was taken. The vital signs icon 504 indicates at a glance what prognosis window is being displayed.

The conditions indictor 506 for each prognosis screen will provide an indication of the most recent scan in comparison with an expected average value for a given user. For example, a temperature's vital sign is illustrated in FIG. 5B as having the condition indication of high due to a measured value of 101° F.

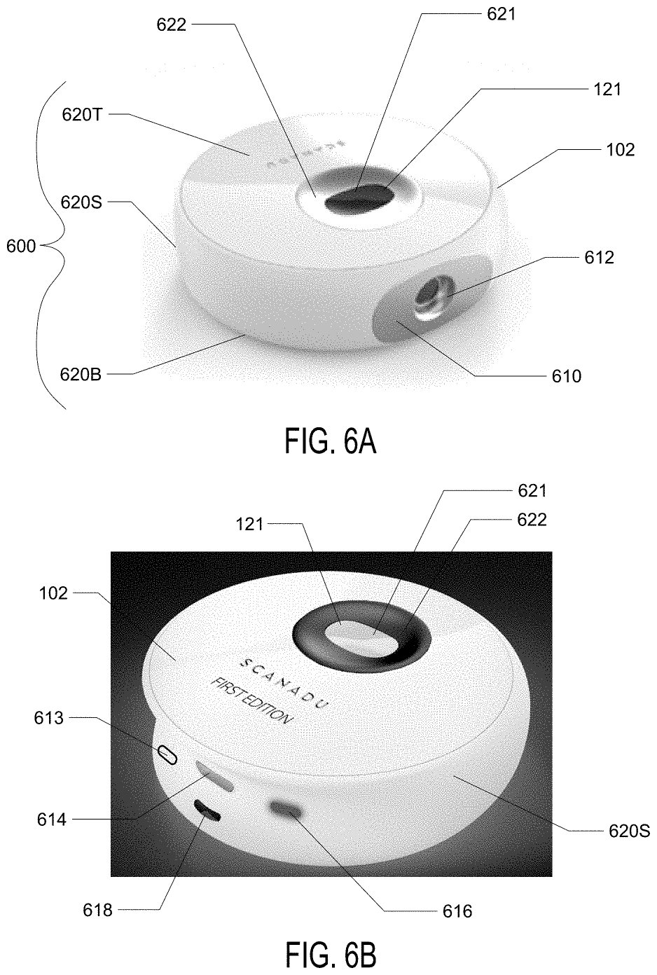

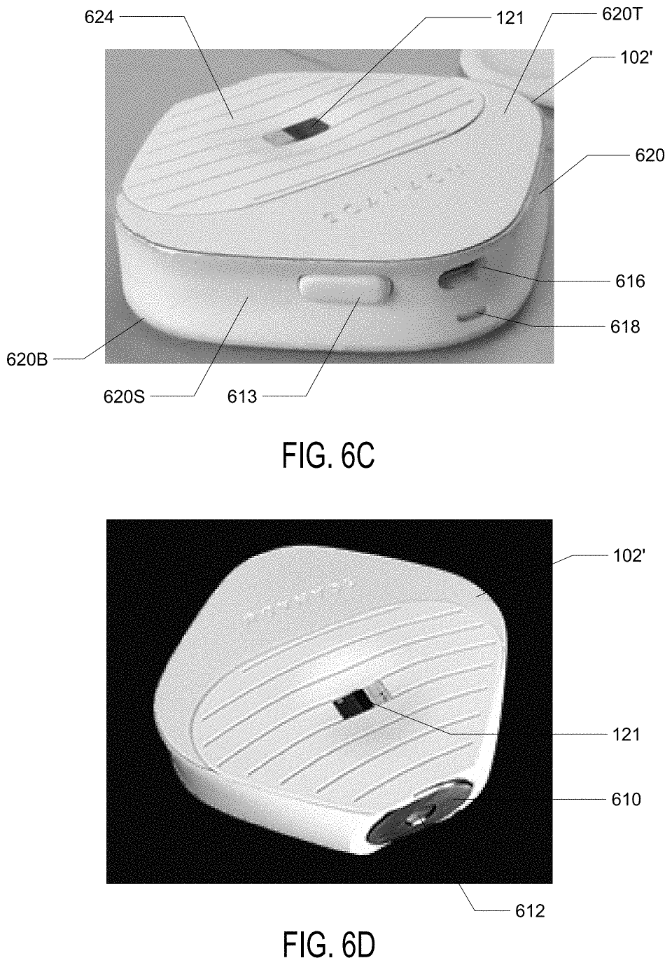

In the vital signs bar 510 the measurement indicator 512 and the vital signs icon 511 may be highlighted to indicate which prognosis screen is being illustrated at a glance. Referring now to FIG. 6A-6D, respective use of the portable wireless vital signs scanner 102 are illustrated. In FIG. 6A, a top front perspective view, the wireless vital signs scanner 102 includes a front electrode 610, and a front sensor 612 on a front side. The front electrode 610 is pressed against the user's forehead, preferably at the temple, in order for the scanner 102 to make an electrical connection to the body of the user.

In one embodiment the scanner 102, a top sensor window 621 and a top electrode 622 are provided in the topside of the scanner 102. A top sensor 121 may be located underneath the top sensor window 621 to obtain a vital signs measurement from a user's finger that may be pressed on top of the window 621. A top electrode 622 may be used to form an electrical connection to a user's finger and complete a circuit of the user's body such as illustrated in FIG. 1A.

The housing 600 of the vital signs scanner 102 may generally be circular shaped and include a circular top housing 620G, a circular bottom housing 620B, and a hollow cylindrical surface 620S. The side cylindrical ring 620S may be concave, or convex over a portion of the surface. Alternatively, the cylinder side surface 620S may be a toroid shape over a portion of its body.

In FIG. 6B, a top back perspective view of the wireless vital signs scanner 102 is illustrated. The wireless vital signs scanner 102 illustrate various aspects of the invention in the side cylindrical surface 620S. The wireless vital signs scanner 102 includes a power button 613, a serial port connector 614, an optional wireless connection LED 618, and a power light-emitting diode 616. The power button 613 may be pressed to power the wireless vital signs scanner 102 on. The serial port connector 614 may be a micro universal serial bus connector to allow a micro USB cable to plug thereto. The micro USB port may provide an external power source to charge the rechargeable battery within the wireless vital signs scanner 102 and also may serve as a wired data port for updating firmware or transferring data to a computer or storage device. The optional wireless connection light-emitting diode 618 provides a visual indicator that the wireless vital signs scanner 102 is coupled to the wireless personal portable multi-function device 104 over its wireless communications channel 103A as illustrated in FIG. 1A. The power light-emitting diode 616 provides an indicator that the wireless vital signs scanner is powered on by the power button 613.

In FIG. 6C, a vital signs wireless scanner 102′ is illustrated having a generally diamond shaped body housing 620. In this case the housing top 620T and the housing bottom 620B generally have a diamond or a square shape to match that of the side cylindrical surface 620S. The top or bottom housing portion 620T may each include a gripping surface 624 with corrugations or channels so that a user may comfortably and securely hold the wireless vital signs scanner 102′. The gripping surface 624 may be formed of a conductive material to aid the top and or bottom electrodes in forming an electrical connection to a user's body.

Referring now to FIG. 6D, a top front perspective view of the wireless vital signs scanner 102′ is illustrated. The wireless vital signs scanner 102′ includes the front electrode 610 and front sensor 612.

While the electrode 622 and the gripping surface 624 are illustrated in the top housing 620T, they may also be implemented in the bottom housing portion 620B instead of the top. Instead of an index finger making a connection with a top electrode 622, a thumb finger may couple to a bottom electrode (not shown) to provide a larger surface area contact to the body in the bottom housing portion 620B.

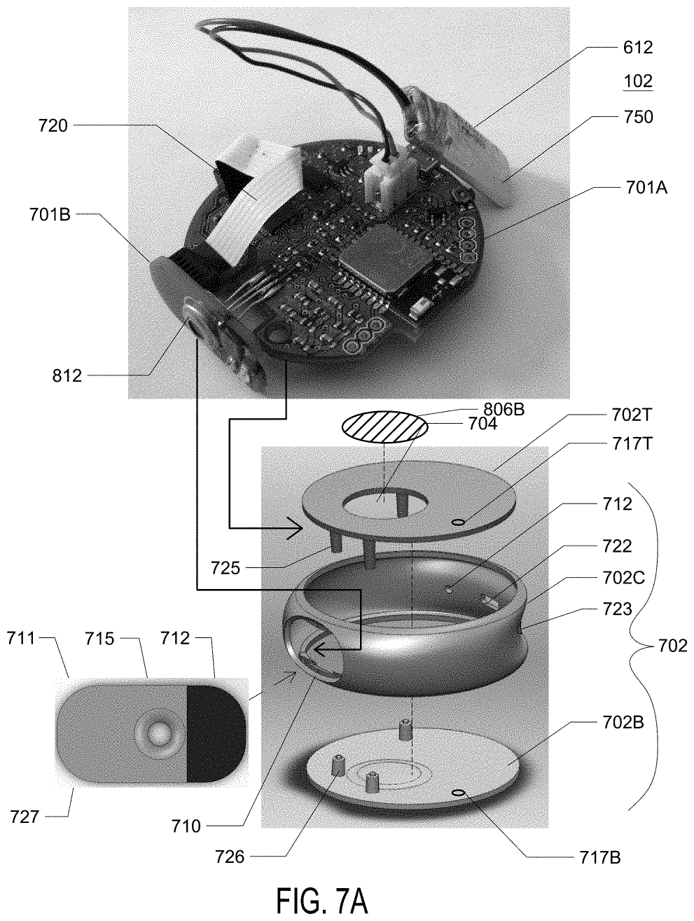

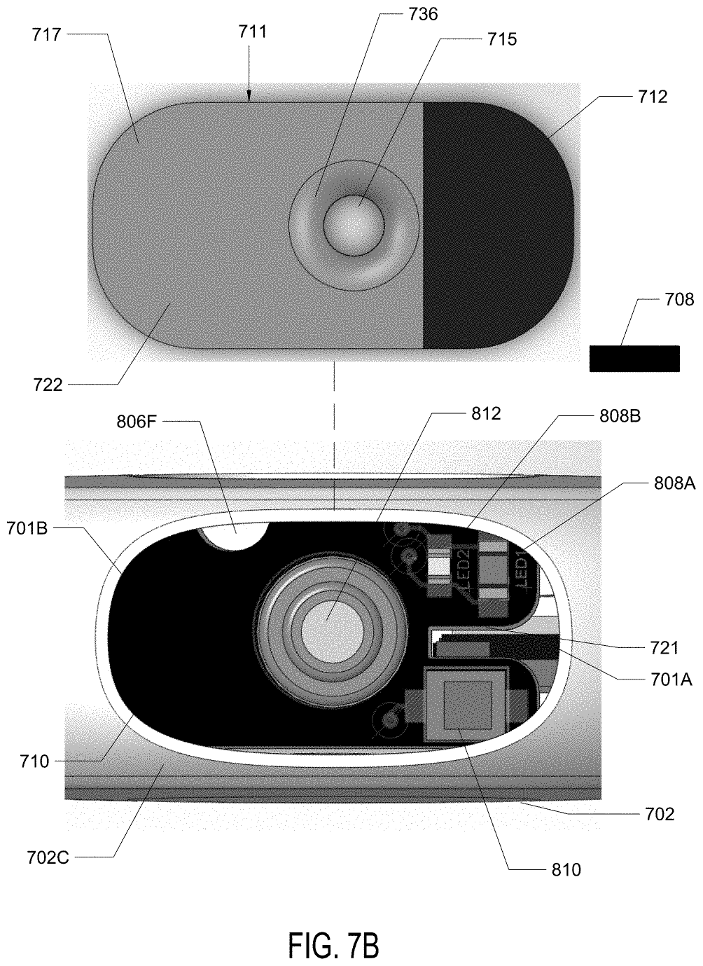

Referring now to FIG. 7A, an exploded view of the wireless vital signs scanner 102 is illustrated. The exterior components of the wireless vital signs scanner 102 are formed of parts that can be wiped clean by a damp towelette or a disinfecting wipe. In this manner, the scanner 102 may be shared by users in a family with less worry about spreading bacteria and germs. Each user may have a personal profile or preferences stored in the scanning software application 140.

The wireless vital signs scanner 102 includes a main printed circuit board 701A and a daughter printed circuit board 701B coupled perpendicular to the main circuit board 701A. Because the scanner 102 is wireless, it includes a rechargeable battery and a connector port to which a cable may connect to recharge the battery. Preferably the battery may be charged in an hour or less. If the scanner 102 is used a few times a day, the charge of the rechargeable battery may last about a week. The main print circuit board 701A, the daughter printed circuit board 701B, and the rechargeable battery form an electronic sub-assembly 701.

The electronic sub-assembly 701 is inserted into a housing 702 of the vital signs scanner 102. The sensors on the front daughter board 701B are aligned into a front sensor opening 710 in the side housing ring 702C of the housing 702. A ribbon cable 720 electrically connects the front daughter board 701B to the main print circuit board 701A. A sensor 812 in the front daughter board 701B includes electrical leads that are coupled to the main printed circuit board 701A.

The main printed circuit board 701A is inserted into the housing ring 702C so that a serial bus connector 612 aligns with the connector opening 722 and the front sensor 812 is aligned into the front sensor opening 710. A top/bottom electrode 806B covers over an opening 704 and is electrically coupled to the main printed circuit board 701A and an ECG circuit mounted thereto.

The housing 702 of the wireless vital signs scanner 102 includes a top housing portion 702T with a top electrode 806B, a side housing ring 702C, and a base housing portion 702B. The orientation of the housing 702 for the scanner 102 may be altered such that the housing base 702B becomes the housing top 702T and the housing top 702T becomes the housing base 702B with a bottom electrode 806B to couple to a thumb. Electrodes may also be in both the housing base 702B and the housing top 702T to provide a lower resistive coupling to the user's body.

The top housing portion 702T includes a microphone opening 717T and a plurality of posts 725 and an electrical sensor opening 704. The housing base 702B may include a microphone opening 717B and a plurality of pillars 726 that can interface to the posts 725 when the housing is assembled together about the printed circuit boards.

The wireless vital signs scanner further includes a front cover 711 to fill in the front sensor opening 710 in the side housing ring 702C. The front side cover 711 includes a plastic cover portion 712 and a front electrode portion 727 with a lens 715 transparent to thermal wavelengths to allow the sensor 812 beneath it to capture a measure of temperature. The plastic cover 712 is also transparent to various wavelengths of light that are used by the vital signs sensors. The front electrode portion 727 of the front cover 711 is formed of a conductive material, such as stainless steel metal, to form a circuit when pressed up against the user's body at the forehead, finger, chest or elsewhere. The shape of the front electrode 727 can vary with the shape of the wireless vital signs scanner 102.

The side housing ring 702C includes one or more LED openings 712 to receive the power light-emitting diode 616 and the optional wireless connection light-emitting diode 618. The side housing ring 702C further includes a power button opening 723 through which the power button 613 may extend.

FIG. 7B illustrates a partially assembled wireless vital signs scanner 102. Through the front opening 710 in the side housing ring 702C, the front daughter printed circuit board 701B includes a slot opening 721. The slot opening 721 may be used to receive a shade 708 that separates the LEDs 808A-808B from the photo diode 810. The shade 708 deters light emitted by the LEDs 808A-808B from directly being impinged onto the photo diode 810. The wire (not shown in FIG. 7B) from the front electrode 727 may inserted through the opening 706 and then coupled to the main PCB and the ECG circuit. The front cover 711 can then assembled to cover over the front opening in the side housing ring 702C of the housing.

The daughter printed circuit board 701B is arranged to be substantially perpendicular with the main printed circuit board 701A. As previously discussed, the front cover 711 includes a transparent cover portion 712 and a metallic conductor portion 727, and the lens 715. The transparent cover portion 712 covers over one or more light-emitting diodes 808A-808B generating various wavelengths of light, and a photo diode 810 that receives various wavelengths of light. The light generated by the light-emitting diodes 808A-808B is shined onto the user's forehead and reflected back to the photo diode 810. Light with known time periods may be generated by the light emitting diodes (LEDs) 808A-808B with different wavelengths and radiated onto a user's forehead. The reflection is detected by the photo diode 810 to form an electrical signal that is analyzed. In this analysis of the signal generated by the reflected lights of different wavelengths, a measure of oxygenation in the blood stream may be generated.

The front side cover 711 includes the transparent lens 715 with a center aligned into the optical axis of the front side sensor 812 so that additional vital signs measurement may be made from the forehead of the user. An opening 706 in the daughter board 701B allows a wire to pass through from the front electrode 727 and be coupled to a wire trace on the main PCB that is coupled to the ECG circuitry mounted thereto. When pressed against the user, the metallic electrode portion 727 of the front side cover 711 makes an electrical contact to the forehead or other body portion of the user. An insulating ring 736 under the electrode portion 727 of the front side cover may be used to isolate any metal of the infrared thermometer 812 from the electrode portion 727.

FIG. 8A illustrates a functional block diagram of electronic circuitry 800 within the portable wireless vital signs scanner 102. The personal portable wireless vital signs scanner 102 associated with a given user profile stored in the user data of the wireless personal multifunction device 104. The wireless communication channel 103A between the scanner 102 and the multifunction device 104 may be a secure connection with information passed between each. The devices are typically paired to each other by a code so that no other wireless device may utilize the wireless communication channel 103A. A different wireless communication channel 103B may be generated between the vital signs scanner 102 and a personal computer 150, for example. Each of the wireless communication channels 103A, 103B may be a Bluetooth communication channel, for example, in which case the signal strength between each over a Bluetooth communication channel is relatively short with a limited distance over a range between zero and twenty-five feet, for example.

Referring now to FIG. 8A, electronic circuitry 800 of the portable wireless vital signs scanner 102 includes a processor 840 at the heart of the system. The processor 840 may be a reduced instruction set processor operating with embedded operating system software. In one embodiment of the invention, the processor is an ARM processor operating with MICRIUM's embedded real time operating system (RTOS).

To provide the wireless communication channels 103A, 103B, a wireless radio 870 is coupled to the processor 841. The wireless radio 870 is coupled to an antenna 871 that could be internal, as part of an overall radio system, or external to the wireless radio 870. An optional light emitting diode 848, used as a wireless connection indicator, is coupled to the wireless radio to indicate a successful pairing with the personal portable wireless digital multifunction device 104. To scan for vital signs over a period of time such as 10 seconds, the electronic system 800 includes an infrared thermometer 812, an accelerometer 885, a pulse oximetry sensor and a pulse oximetry circuit 880, and analog electrocardiogram circuitry 860. Coupled to the electrocardiogram circuitry 860 is the bottom or top electrode 806B, the front electrode 711, bottom/top electrode connection, and the front electrode connection 806F. As shown in FIG. 1A, a portion of a human body is coupled to the front electrode 711 and the top/bottom electrode 806B to form a circuit.

The pulse oximetry circuit 880 is coupled to a pair of light emitting diodes 808A-808B. Each of these emit light patterns that are reflected off of the user's forehead internally. The reflected light is captured by a photodiode 810 and coupled to the circuit 880. That is, incident light 891 from the light emitting diodes 808A-808B reflects internally off the user's head 116 as reflective light 892 which is received by the photodiode (PD) 810.

The infrared thermometer 812 detects the surface temperature of a use's forehead (or elsewhere) by measuring thermal radiation (referred to as Blackbody radiation) 813 emanating from the head 116 (or other body portion to which the scanner is pressed) of a user.

To power the circuits in the system 800 of the personal portable wireless vital signs scanner 102, a rechargeable battery 850 and a voltage regulator and battery charge controller 854 are coupled together into the circuits in the system 800 when the switch 852 is closed. The battery charge controller 854 is coupled to power pins of a serial connector 856 to receive an external DC voltage supply. The external voltage supply may be used to recharge the battery and power the system 800 when it is connected. The rechargeable battery 850 may hold a charge for a period of seven days, even while scanning multiple times during each day, due to the low power consumption of the circuitry and the limited period of time needed to perform a scan of the vital signs of a user. That is, the vital signs scanner 102 is not expected to be continuously powered on during a day, but powered up periodically to perform the scans as needed.

The processor 840 may include a processor memory 841 to store system instructions to control the circuitry in the system to obtain the scans and process the information obtained through those scans into a proper user format. To store the user data from each of these scans, a nonvolatile memory 844 is coupled to the processor 840. The nonvolatile memory 844 may be soldered to a printed circuit board with the processor 840. In an alternate embodiment of the invention, a connector 845 is provided so that the nonvolatile memory 844 is a removable memory card so that a user's data may be transferred from one scanner to the next, if needed.

A power LED 851 may be coupled to the processor 840 to provide an indication that the electronic system 800 is powered up. The system can be manually shut down via the scanning software application 140 so that the scanner 102 powers off. However, the scanner 102 can also automatically shut off after a predetermined period of time to conserve power and a charge on the rechargeable battery 850. The user then just needs to press the power switch 852, once again, to turn the system back on and scan for vital signs of a user.

The processor 840 includes one or more analog digital convertors 842 in order to receive analog signals from the infrared thermometer 812, accelerometer 885, pulse oximetry circuits 880, and ECG analog circuits 860. Electronic system 800 may further include a stereo microphone 875 consisting of a top microphone 875T and a bottom microphone 875B each coupled to a stereo microphone amplifier 874. The stereo microphone amplifier may have its own analog to digital converter, or the processor's analog digital convertor 842 may be used to convert analog signals into digital signals. For example, an ECG analog signal may be converted into digital signals with the analog digital convertor 842 of the processor. The stereo microphone 875 captures audio signals near the wireless vital signs scanner 102. The accelerometer 885 captures movement of the portable wireless vital signs scanner 102.

The combination of the audio information and the movement information may be utilized to determine the quality of the scanning information being obtained by the vital signs capturing circuitry. For example, the stereo microphone 875 may be used to capture noise from a user talking and plot that on a graph indicating noise spikes, or noise lines 330, such as shown in FIG. 3A. This provides feedback to a user about the quality of the scan at these intervals. The accelerometer 885 and the motion information may be similarly used to make a judgment about the quality of the vital signs scanned information being captured by the vital signs circuitry of the infrared thermometer 812, the pulse oximetry circuits 880, and the ECG analog circuits 860.

The microphones 875 in the portable wireless scanner 120 may also used to capture body sounds such as shown in FIGS. 1E-1F and store the captured body sounds in memory 844 as a potential symptom of a medical condition of the users body. For example, heart beat sounds may be captured by the microphones 875 when the scanner 102 is positioned against skin of the chest near ones heart, as is illustrated in FIG. 1E. As another example, lung or breathing sounds of air entering and exiting ones lungs may be captured by the microphones 875 when the scanner 102 is positioned against skin of the chest near a lung in ones body, as is illustrated in FIG. 1F.

To further optimize scanning results, scan quality algorithm monitor the vita signs scanning process and can provide feedback (visual and/or audible) to the user, such as through the multifunction device 104.

An optional audible sound generator 847 in the scanner 102 may be coupled to the processor 840 to provide audible user feedback to the user during the scanning process. The user feedback may help the user to perform better vital signs scan with the wireless vital signs scanner 102 and acquire a higher quality of vital signs measurements. The audible sound generator 847 may generate alert sounds indicating when the scanning process begins and ends. It may also generate an error signal indicating to the user that he is not properly using the scanner 102 and look for instructions on the device 104.

Referring now to FIG. 8B, a functional block diagram of the electronic circuits 800 are shown mounted onto the main printed circuit board 801A and the daughter printed circuit board 801B. FIG. 8B also illustrates alternate locations for electronic circuits in the system 800 for alternate embodiments of the vital sign scanners 102, 102′.

A slot 803 in the daughter printed circuit board 801B receives a shade device 708. Light emitted by the LEDs 808A-808B is shaded by the shade device 708 so that it may not directly impinge onto the photo diode 810 in the daughter PCB 801B. Reflected light, reflected off the user's body, is desirable to be captured by the photo diode 810.

Wire leads 830 of the IR sensor 812 and the front electrode contact 806F are coupled to pads 831of the main printed circuit board 801A. First and second LEDs 808A-808B and the photodiode 810 are coupled to connector 821 by conductive traces 819B on the daughter printed circuit board 801B.

The main printed circuit board 801A has a plurality of wire traces 819A coupling circuits mounted thereto together. The daughter printed circuit board 801B includes a plurality of traces 819B coupling circuits mounted thereto to connector 821. A ribbon cable 820 is used to couple signals between the daughter memory card 801B and the main printed circuit board 801A for the oximetry circuit 880. The oximetry electronic circuit 880 is coupled between the connector 822 and the processor 840 on the main printed circuit board 801A.

In accordance with one embodiment of the invention, if the oximetry sensors are moved to a top portion of the housing to sense oximetry through a finger, with the RLEDs 801A′-801B′ and the IR photodiode 810′, the oximetry circuitry may be moved to the opposite side coupled between the processors 840 and the LEDs 808A′-808B′, 810 and mounted in the top portion of the housing. The bottom or top electrode 806B is formed of stainless steel to provide a good connection to either a thumb finger or an index finger. The electrode 806B is coupled to a connector 823 and to the ECG circuitry 860 on the main printed circuit board 801A.

The main printed circuit board 801A includes the processor 840, the wireless radio 870, the microprocessor memory 841 (either internal or external as shown mounted to the printed circuit board), an accelerometer 885, an amplifier 874, oximetry circuitry 880, 880′, user memory 844, and battery charge circuit 854.

Top and bottom microphones 875T and 875B extend out from the main printed circuit board 801A by ribbon cables so that they may be mounted into the respective openings in the housing top and housing bottom. The microphones 875T, 875B may be coupled to the amplifier 874 which in turn may couple audio signals into the microprocessor 840. Mounted to the main printed circuit board is the power LED 851 and the connection LED 848. Further mounted to the main printed circuit board is a power on/off switch 852 coupled to the voltage regulator battery charge circuit 854 to signal for it to turn power on or off to components with the scanner 102. Additionally, mounted to the main printed circuit board 801A is a serial connector 856 coupled to the microprocessor. In one embodiment invention, the serial connector 856 is a micro universal serial bus connector. An optional audible sound generator 847 may be mounted to the main PCB 801A and coupled to the processor 840 as shown. To avoid interference, the sound generator 847 may be positioned away from the microphones 875.

Main printed circuit board 801A includes a plurality of openings 826 that receive the pillars 725, 726 of the housing top 702T and housing base 702 B.

The daughter printed circuit board 801B includes a connector 821, light emitting diodes 801A-801B, and a photodiode 810 mounted thereto. The IR sensor 812 is inserted through a hole in the daughter PCB 801B, attached thereto with an adhesive, and supported thereby. The front electrode 806F around the IR sensor 812 is attached with an adhesive to the daughter PCB 801B for support.

The ribbon cable 820 couples signals of the light emitting diodes 801A-801B and the photodiode 810 regarding oximetry between the daughter board 801B and the main printed circuit board 801A for the oximetry circuit 880. With the terminals 830 of the IR sensor 812 coupled to the pads 831 of the main PCB 801A, signals of the IR sensor 812 regarding temperature are coupled into the processor 840. With the terminals 830 of the front electrode 806F coupled to one or more pads 831 of the main PCB 801A, signals of the ECG circuit 860 to measure heart activity (e.g., heart rhythm, heart rate, etc.) may be coupled into and out of a users body.

Thus, the personal portable wireless vital signs scanner integrates a plurality of sensors and a controller/processor together to synchronously obtain a plurality of vital signs at different times during a users day. Despite the integration of multiple sensors and a controller/processor into the scanner, the vital signs scanning device has a relatively low production cost. The integration with a ubiquitous consumer electronic device pre-owned by many users, the personal wireless multifunction device (e.g. smartphones, tablets, etc.), to display the vital signs data with vital signs scanning software, also keeps the costs low of the overall personal vital signs scanning system. The low costs of production of the vital signs scanner can allow lower retail pricing and higher volume of sales, enabling an average consumer to afford the vital signs scanning system to personally scan and monitor trends of their vital signs for as an important part of preventive medical care of their own bodies.

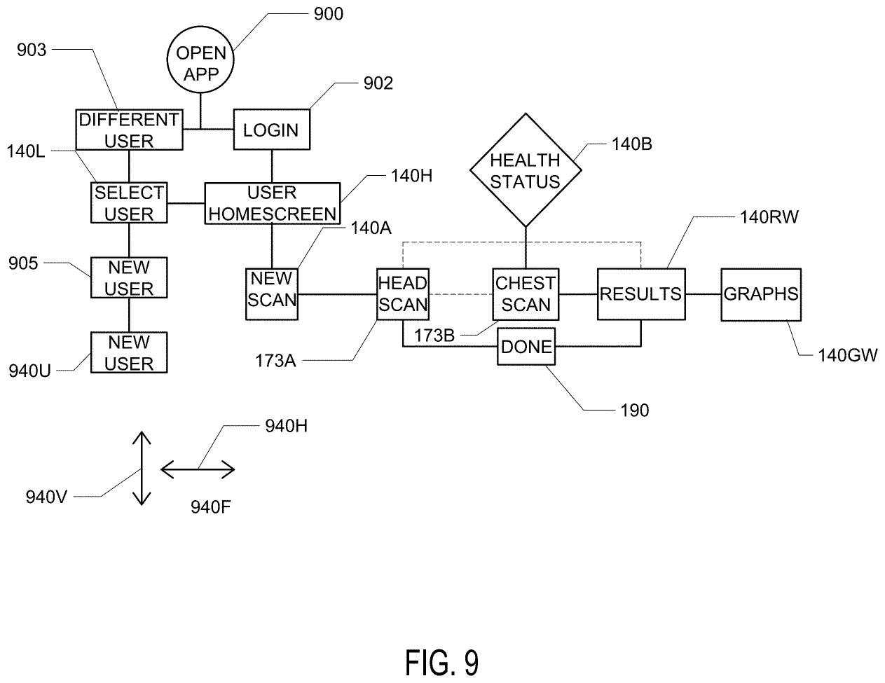

Referring now to FIG. 9, a diagram illustrating an exemplary hierarchy of windows provided by the scanning application software 140 is illustrated. A variety of vital sign scanning user interface windows of the scanning application software 140 have been described. The vital signs scanning application software 140 executed by a processor provides a user interface hierarchy of the vital sign scanning user interface (VSUI) windows. For example after the vital sign scanning application is opened at process 900, a scanning login button 902 may be presented to the user by the scanning application software 140. If the user is properly selected he chooses the login button to transition to a user home screen 140H. In the user home screen, a user inputs his login identification and password to gain access to personal vital signs scan data stored in the device 104. If the user is a different user a different user button 903 may be selected or a horizontal swipe finger gesture 940H may be used to go to a select user window 140L. If the user is not listed and is a new user, the select user window 140L may have a new user button 905 that jumps to a new user window 904U that is displayed to the user. In the new user window 940U, the new user may input his login user ID and password that he desires to use with the scanning software application to identify his personal vital signs scan data. Other information, such as sex, height, weight associated with a time and date may be entered by the user. As the days and/or years go by, the user may update this information in the profile so that the vital signs scanning system better knows what conditions might occur for the given user. The login and profile windows can also allow the scanning system to be shared with other users in a family. After logging in with user ID and password through the home screen, the scanning system application may display the initial scanning window 140A.

By using a horizontal finger gesture 948 over the scan type slider 171, the user may select a head scan 173A, a chest scan 173B, the results window 140C, or the graphs window 140D. If at any point in time the user feels the need to terminate the scanning process, the done button 190 in the user interface may be selected. By means of a vertical finger gesture 940 V in the scanning window 140A during a head scan 173A or chest scan 173B, or the results window 140C, the health status window 140B may be displayed. Alternatively a health status button 180 may be selected to display the health status window 140B.

Each of the screens/windows/slides of the vital signs scanning application may be navigated by pressing one or more virtual graphical buttons (e.g., back, done) and/or making one or more finger gestures 940F (e.g., vertically up/down 940V, horizontally left/right 940H) dragged across a touch screen. A navigation bar may alternatively be provided with navigation buttons to navigate between selected windows. The menu button may also be used to navigate to different windows. In other cases, pressing a button displays a different screen/window/slide such as the done button.

After scanning is completed, the scanning application software can automatically display the results window 140C. Additional buttons in the results window 140C may be used to navigate to various graph windows 140D, such as the temperature graph window shown in FIGS. 4A-4B. Additional buttons in the results window 140C may be used to navigate to various prognosis windows shown in FIGS. 5A-5B. In this manner, vital signs data and information can be displayed to the user in various ways.

The scanning software application 140 includes a number of instructions and routines that are executed by a personal wireless multifunction device 104. The personal wireless multifunction device 104 may include a smart phone, such as an APPLE IPHONE 5, IPHONE 4S, or SAMSUNG GALAXY S III, that supports Bluetooth Smart wireless communication with the vital signs scanner 102 and cellular wireless data communication, Wi-Fi wireless data communication, and/or Ethernet wired data communication over a communication network. To help everyone use the multifunction device 104, assistive technology may be added to the scanning software application 140.

The significant software routines of the scanning software application 140 include a scan procedure controller based on scan quality algorithm, UI implementation, wide area network interfacing to cloud services, scan results interpretation, and trend charting.

### Cloud Data Service

The scanning device 102 detailed above allows a patient to effortlessly scan their vital signs multiple times throughout each day. The user friendly scanning software application 140 running on the multi-function device 104 displays the client's vital signs in an easy to read manner. Combined, the two devices allow a user to take a more hands on approach to monitoring and maintaining their health. Further adding a cloud based/Wide Area Net (WAN) based access and storage system allows even further data analysis for an even more comprehensive health monitoring program.

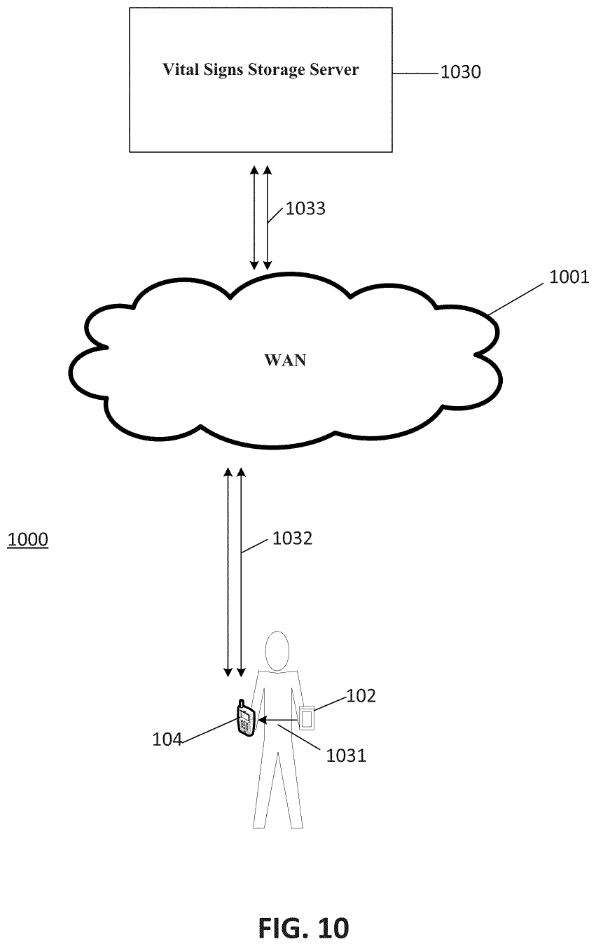

FIG. 10 is a block diagram illustrating an exemplary vital signs cloud system 1000 including a vital signs scanner 102, a multifunction device 104, and a vital signs server 1030 in communication together. A typical user scans their vital signs using the vital signs scanner 102. The raw sensor data is processed by a processor onboard the vital signs scanner into processed vital signs data. The processed vital signs data may then be wirelessly transmitted to the user's multifunction device 104 and displayed by a scanning application running on the multifunction device 104.

The multifunction device 104 may store multiple monitoring sessions. For instance, the multifunction device 104 may store one year's worth of processed vital signs data. Intermittently, the processed vital signs data may be uploaded to a vital signs storage server 1030. For privacy and data fidelity, the processed vital signs data are encrypted to minimize data security risks on the multifunction device before upload. The off-site vital signs storage server may hold multiple years' worth of the user's vital signs data, even a lifetime's worth of data. To upload processed vital signs data from the multifunction device 104 to the vital signs storage server 1030, a secure connection may be made via a wide area network (WAN) 1001, such as a wireless network, e.g., Wi-Fi (any wireless local area network and wireless access point products that are based on the Institute of Electrical and Electronics Engineers 802.11 standards) or cellular networking; a wired network; and/or a combination of a wireless network and a wired network.

In FIG. 10, a wireless communication connection or channel 1032 may be formed between the multifunction device 104 and the network 1001 for bidirectional data communication to/from the vital signs storage server 1030. A communication connection or channel 1033 may be formed for bidirectional data communication between the network 1001 and the vital signs storage server 1030. Vital signs data stored in the multifunction device 104 may be uploaded to the network 1001 and then to the storage server 1030. Historical vital signs data may be downloaded from the storage server 1030 over the network 1001 to the multifunction device 104 by the same the communication connections or channels 1032,1033. Data flows in both directions (bi-directional) over the communication connections or channels 1302,1033 as indicated by the parallel double-headed arrows.

To protect privacy, the multifunction device 104 encrypts the data that is transmitted to the vital signs storage server 1030 over the network 1001. The multifunction device 104 decrypts encrypted data that is received from the vital signs storage server 1030 over the network cloud 1001. To enable decryption of the encrypted data on another device, proper authorization is needed. Data flow back to the multifunction device 104 from the server 1030 may include, for example, summary data of several months or even years of the user's vital signs.

### Personal Vital Sign Curves

A single page of a novel does not tell a complete story. Similarly a single day's scanning result does not accurately portray a patient's medical history. The aggregation of a patient's scanning results throughout an extended period of time may be more comprehensive and thereby more useful to both the user and the user's medical professional. Accumulated vital signs data may be offloaded to cloud servers, such as the server 1030 shown in FIG. 10. The accumulated vital signs data may be processed to show trends in the user's vital signs over time. The accumulated vital signs data for a given user may also be compared with groups of users with similar traits to check for normal and/or abnormal health conditions. The accumulated vital signs data for a given user may also be shared with the user's medical professional to verify normal and/or abnormal health conditions.

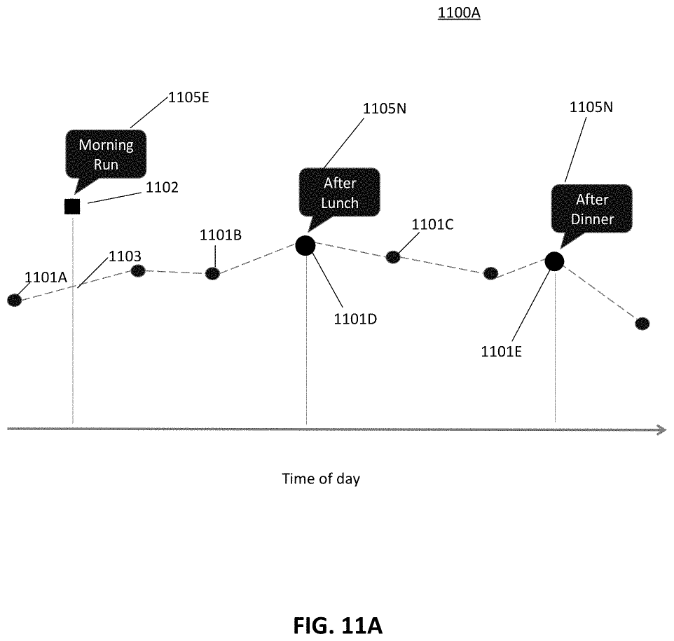

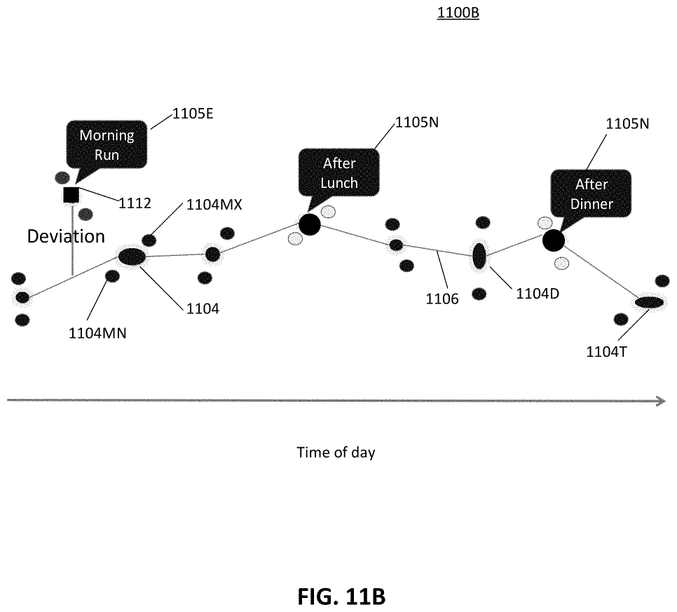

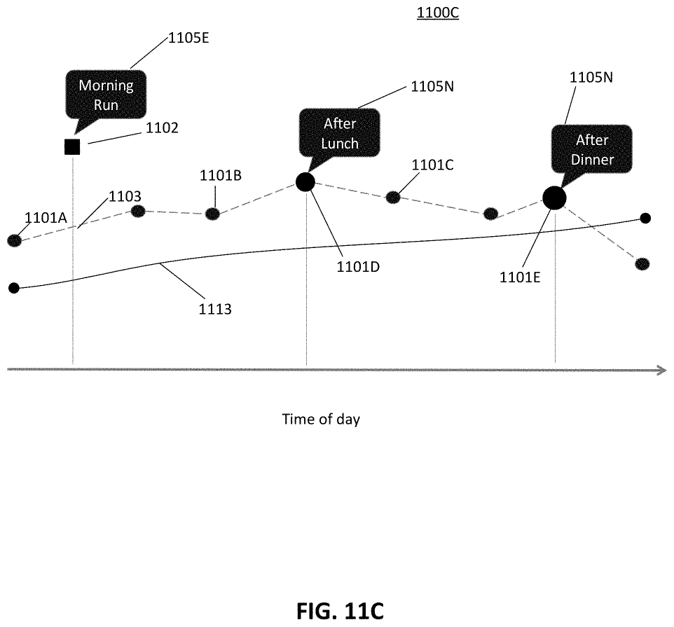

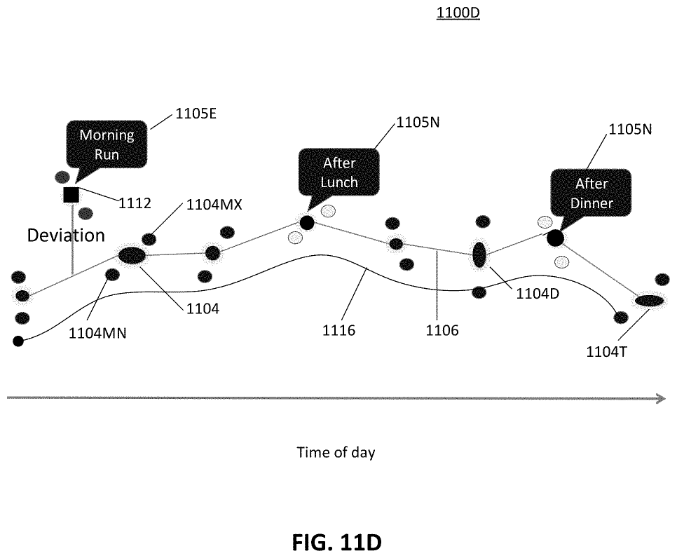

The basic health of a user can be represented by reference vital sign curves of multiple vital sign measurements. Every user is different so their personal vital sign curves will be different. A tagged reference vital sign curve can be represented as a canonical vital sign curve plus tagged deviations, such as shown in FIG. 11B. Everyone will have his/her own canonical vital sign curve as the base temporal representation of one vital sign measurement.

Easy-to-use vital sign scans that may be done anytime and anywhere, motivates the average user to start building personal vital signs curves that lay the foundation of their basic health. The ease of using the vital signs scanner will encourage a healthy routine of constant monitoring. Over time the accumulated vital signs data may be processed to show trends.

To build useful reference vital signs curves, the vital signs are aggregated according to time. FIG. 11A illustrates a basic vital signs chart 1100A over a single twenty-four hour period. The x-axis indicates the time of day and the y-axis indicates the magnitude of the given vital sign being displayed. Plotted points represent discrete vital sign scans at different time points during the same day. Events may occur during the day that can effect the vital signs data during a vital signs scan. Some events may be extraordinary that occur infrequently and not considered in forming the curve 1103. Other events may be normal or ordinary, occurring regularly or frequently, and are to be considered in forming the basic health curve 1103.

The plotted points on the chart may have different shapes (e.g.,. circular, square, or triangular) or different colors (e.g., blue, red, green) to indicate additional information about a plotted point. Circular plotted points, such as points 1101A, 1101B, and 1101C, may represent normal discrete vital sign scans at different time points during the same day. Square plotted points are also vital sign scans captured at a different time point during the same day, such as square plotted point 1102. The square plotted points, such as square plotted point 1102, may be tagged with an extraordinary event tag 1105E, such as a morning run, during which time point the vital signs scan may be taken and substantially deviate from normal.

Other ordinary or normal occurring daily events may be tagged with a normal event tag 1105N after which vital signs are taken, such as when vital signs are taken after eating a meal. The circular plotted point for vital sign scans taken after such normal tagged events, such as plotted points 1101D-1001E, may be colored differently, shaded differently, or shaped differently than other circular plotted points for which no event is tagged. These points are still considered to be normal and plotted on the curve 1103. For example, vital signs may be taken after lunch and/or after dinner and tagged as a normal event with a normal event tag 1105N for plotted points 1101D-1101E, such as shown in FIG. 11A.

The curve 1103, a one day vital sign curve, may be plotted between the circular or normal plotted points to show a selected days trend for a selected vital sign of the user. The curve 1103 represents the daily trend of a single vital sign, such as body temperature for example. For different vital signs, such as blood pressure, blood oxygenation, heart rate, respiration rate, ECG values, etc., additional one day vital sign curves may be generated and displayed to the user by additional user interface screens. While a one day vital sign curve is useful in determining a given days base health condition, historical vital signs data may be useful in determining averages and health trends of the user.

FIG. 11B illustrates a vital signs chart 1100B comprising data from multiple twenty-four hour periods. Historical vital signs data stored in storage devices (e.g., non-volatile memory) of the multifunction device 104 and in storage devices of the vital signs storage server 1030 are used to update a default canonical average curve 1106 for each vital sign. A canonical average curve 1106 may be plotted for a given vital sign that represents an averaging of the user's stored historical vital signs data. The canonical average curve 1106 may be an average of all the scans made by the user. Alternatively, the user may select the period of scans that are to make up the canonical average curve 1106. For example, the user may select a given week, month, year, or range of years of vital sign scans to plot and form the canonical average curve 1106 for each vital sign. Alternatively, a user may select all vital signs scans up to a selected age or all vital signs scans within a range of ages to plot and form the canonical average curve 1106 for each vital sign.

The canonical vital sign curve includes an indication of the standard deviation, as does the averaged tagged scans, e.g., Morning Run. Instead of circular plotted points, oval plotted points 1104 are used to show the standard deviations. The vertical deviation in an oval plotted point 1104 along the curve 1106, such as shown by the oval data point 1104D, indicates the standard deviation in the magnitude of the vital sign value that is taken over the historical scans. The time differences between the scanning sessions that make up the oval data points 1104 along the curve 1106 can also be indicated. A horizontal stretch of the oval data points 1104 indicates the variation in times of day when aggregation of historical scans were taken. For example, the oval plotted data point 1104T illustrates an elongation along the curve 1106, indicating that there is a variation in time (e.g., 30 minutes for vital signs scans and data obtained at 10:00 PM, 10:05PM, 10:15PM, 10:20PM, and 10:30PM that are aggregated together into the plotted data point) when the vital sign scans were taken over the selected period of days, months, or years.

A rectangular plotted point 1112 may also be used to indicate history of similar extraordinary events that are not considered in forming the curve 1106. Similar to the oval plotted points, the rectangular plotted point 1112 may be stretched vertically to show the standard deviation in the vital signs data over the selected history of daily scans. Additionally, the time when the vital signs scan is taken after the same extraordinary event can differ. The rectangular plotted point 1112 may be stretched horizontally to show the time variation in the vital signs data over the selected history of daily scans for the event.

The maximum sign data point 1104MX and the minimum vital sign data point 1104MN associated for each oval plotted data point 1104 may also be plotted about each as shown in FIG. 11B. The maximum sign data point 1104MX and the minimum vital sign data point 1104MN may be vital sign values that influence the oval plotted data point 1104.

A user may tag additional information about each vital signs scan. For example, additional information that may be tagged to the vital signs scans includes where and why a user is taking the vital signs scan. A tag 1105 may be a standard event that occurs every day, such as a meal at breakfast, lunch, and dinner. A tag 1105 may also describe the basic health condition of a user at the time of the scan. If the user is sick, has a fever, or feels dizzy during a vital signs scan, the user can annotate that information in a tag to the vital signs scan. Other health information that may tagged includes medication information, such as a tag for the name and dosage of the medication taken at or near the time of the vital signs scan. Even dietary information may be important information that can be added to a vital signs scan by use of a tag. For example, a user may input tags describing a high protein or high carbohydrate meal before a scan.

To be more specific, each vital sign measurement is presented by a tagged reference curve or a temporal curve with sample points annotated by attributes. As a result, the reference vital curves are tagged curves of vital sign measurements. These are vital signs curves with knowledge tags that capture knowledge in the form of descriptions, categorizations, notes, annotations, or even hyperlinks regarding the scan.

The information in an event tag may provide an explanation for a scan that lies outside the canonical average curve. For example, in FIG. 11B, the square plot point 1102 tagged with the event tag 1105 “Morning Run” lies well above the canonical average curve. A user looking at that scan without the benefit of an event tag may not remember why their vital sign was so elevated during that scanning event. However, with the event tag, a user may review their aggregated vital signs data and filter such deviations. Standard tag events such as morning, lunch and dinner that repeat, for example, can be added to the canonical curve, while other tag events that are infrequent or less repetitive (e.g., one time events) can be excluded from the canonical curve calculation.

Optionally, similar events may even be aggregated and displayed so that all “Morning Runs” for a month or a year could be plotted together in an event tag 1105, such as the event tag 1105 in FIG. 11B. Elevated vital signs outside the normal average for a “Morning Run” event could then alert the user to potential medical problems that may have remained undetected without the event tag system.

In one embodiment of the invention, canonical curves and tagging may be facilitated through a user interface of the vital signs scanning software application 140 that is executed by a processor. Upon initialization, the scanning software application 140 provides a default canonical vital curve without tagged deviation. After each successful scan, the scanning software application 140 asks users for optional tagging information.

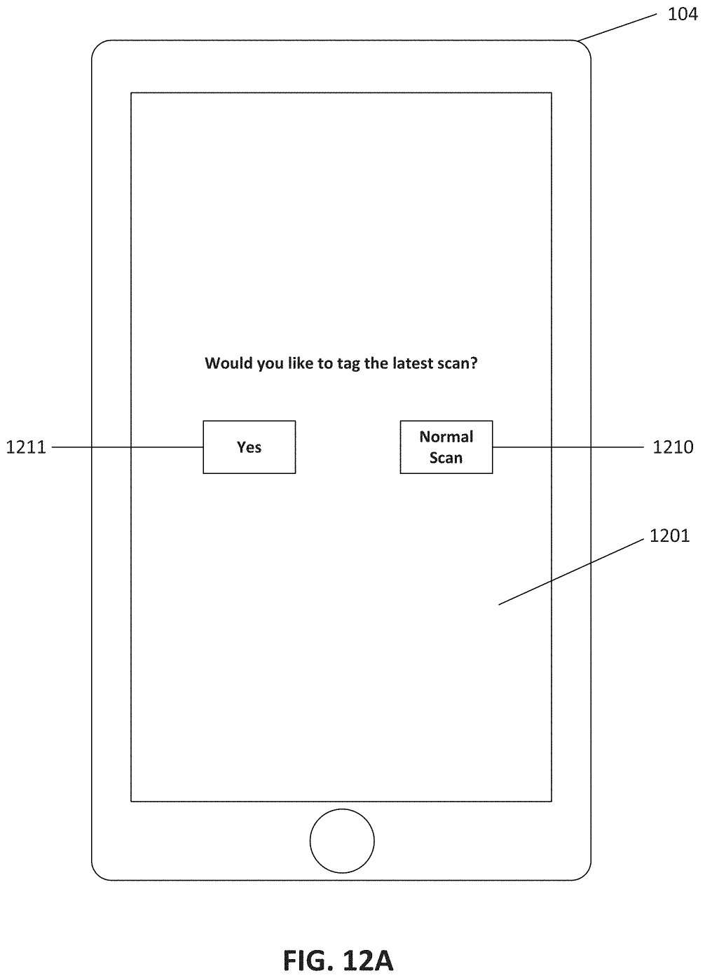

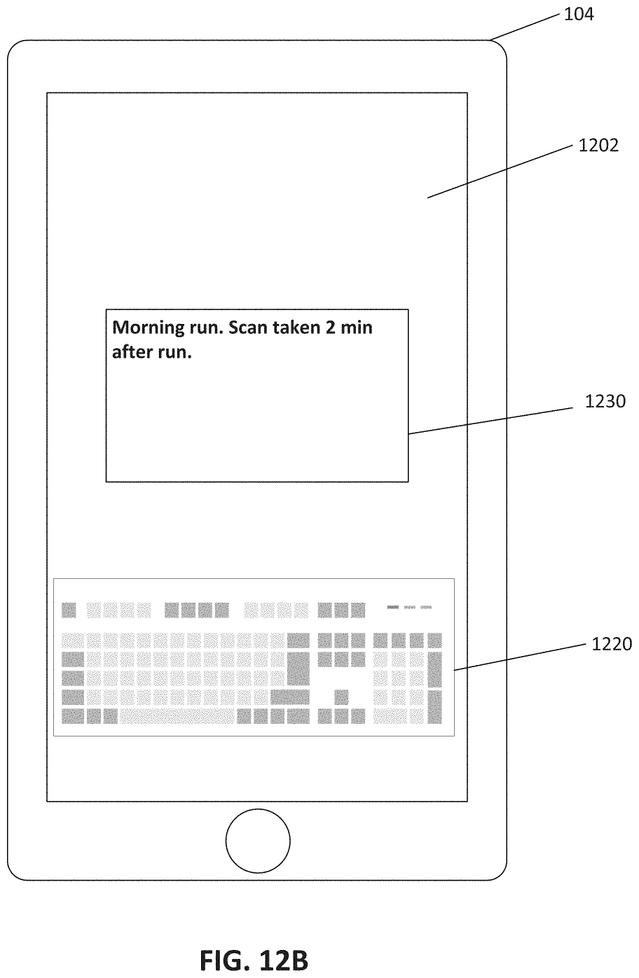

FIG. 12A, illustrates an exemplary initial tagging user interface screen 1201 displayed by the vital signs software application 140 on the multifunction device 104. The software application 140 queries the user through the tagging user interface screen 1201 as to whether they wish to add a tag to the latest vital signs scan. The user can select a decline to tag the vital signs scan by selecting a normal scan user interface button 1210. The user can select to tag the vital signs scan with additional information by selecting the yes user interface button 1211. If the user selects the yes user interface button 1211, another user interface screen is displayed by the vital signs software application 140.

Referring now to FIG. 12B, a tag entry user interface screen 1202 is displayed by the vital signs software application 140 on the display screen of the multifunction device 104. The tag entry user interface screen 1202 includes a virtual keyboard 1220 to allow input of the tag information that is to be tagged to the vital signs scan. A tag information window 1230 displays the tag information that was input and is to be tagged to a vita signs scan.

By not tagging any information or by simply clicking the normal scan button 1210, the software application 140 will understand this is to be a normal vital signs scan with normal sample points, and add the normal scan as an update to the canonical vital curve.

With a tagged scan and tagging information, the canonical vital signs curve will not be updated. Rather, one tagged deviation will be calculated by subtracting the measurements against one canonical point with the same or similar timestamp from the canonical vital curve.

When more and more scanned points are added, the vita signs software application 140 will perform a clustering algorithm to keep the representation of the canonical vital curve and tagged deviations compact. Once enough vital sign data is accumulated, with the user's acknowledgement and authorization, the vital signs software application 140 can start making useful recommendations based on a comparison of the current vital signs scan data from a current vital signs scan with a historical canonical vita signs curve associated with the user.

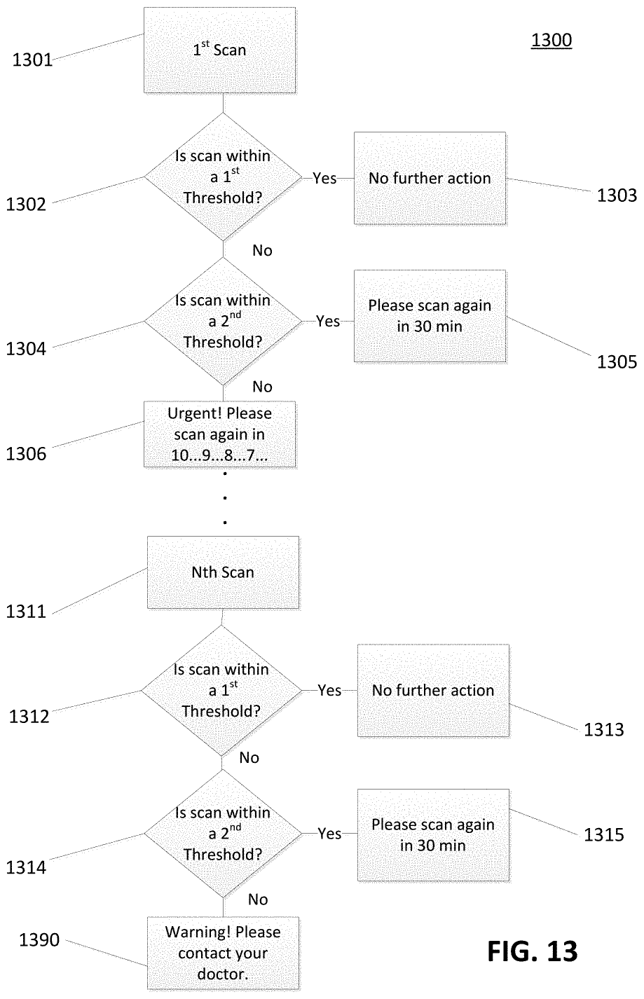

Referring now to FIG. 13, a flow chart of a method 1300 of providing user recommendations during the vital sign scanning process is shown. The vital signs software application 140 may make scanning recommendations to improve vital signs monitoring. In one embodiment of the invention, the software application may assign thresholds that trigger certain recommendations. Alternatively, users may set up the thresholds themselves based on well-known thresholds (e.g., low grade fever at 100 degrees Fahrenheit, high grade fever 104 degrees Fahrenheit, Hyperpyrexia at 106.7 degrees Fahrenheit, constant fever over 24 hours, chronic fever over three days, prolonged fever of 10 days or more) available to the public.

The method 1300 starts with a first vital signs scan 1301. The process then goes to process block 1302.

At process block 1302, a determination is made if the current (tagged) vital signs scan is within a first threshold from the (tagged) reference vital curves. If yes, the process goes to process block 1303 and the user is informed that no further action is needed on their part. With current vital signs data being within the first threshold difference, it indicates a more normal condition of the user with the current vital signs scan. If the current vital signs data is not within the first threshold difference, it may indicate a less normal condition. If the current vital signs data is outside of the first threshold difference from the reference vital sign curves, further investigation is undertaken with the process going to process block 1304.

At process block 1304, a determination is made if the magnitude of the vital signs data associated with the current (tagged) vital signs scan deviates from the (tagged) reference vital curves by more than a second threshold. That is, is the magnitude of the vital signs data within the second threshold value while exceeding the first threshold value. The second threshold could represent for example a maximum set value and/or a minimum set value for the vital sign data. If the vital signs value does not exceed the magnitude of the second threshold, perhaps some event or activity caused it to be temporarily be outside the first threshold. Another scan may be advisable after waiting a predetermined period of time. The process can go to process block 1305.

If the current (tagged) scan data of the most current vital scans deviates from the (tagged) reference curves beyond the second threshold, the process goes to process block 1306.

At process block 1305, the vital signs software application 140 can display a reminder on the display of the multifunction device 104 that the user should take a follow-up scan within a predetermined period of time, such as within the next thirty minutes.

At process block 1306, the vital signs software application 140 can display a warning sign on the display of the multifunction device 104. The vital signs software application 140 can automatically set a timer instructing the user to urgently take a follow-up vital signs scans.

The vital signs software application 140 can repeatedly step through the scanning and the determination processes N times. At process block 1311, an Nth vital signs scan is performed with the vital signs scanner and multifunction device. At process block 1312 a determination is made if the magnitude of the vital signs data of the current vital signs scan exceeds or is within the first threshold. At process block 1314, a determination is made if the magnitude of the vital signs data of the current vital signs scan exceeds or is within the second threshold. If the vital signs scan results continuously exceed the second threshold difference or get exceedingly worse, the process goes to process block 1390. If the vital signs scan results continuously decrease and fall below the first threshold difference, then the process goes to process block 1313.

At process block 1313, an encouraging icon e.g. smiley face or thumbs up may be displayed with no further actions recommended.

At process block 1390, the vital signs software application 140 can display another warning on the display of the multifunction device 104. The vital signs software application 140 displays an urgent recommendation that the user immediately contact a doctor or visit an emergency room.

If the vital signs scan results fluctuate between the first threshold difference and the second threshold difference, the software application 140 can display a recommendation that more follow-up scans be performed with the vital signs scanner within a time threshold or that a primary care practitioner be contacted for more information about the user's health.

### Group Vital Sign Curves

With cloud storage of vital signs data by the vital signs storage server 1030, such as shown in FIG. 10, and a user interface provided by the vital signs scanning software application 140 of the multifunction device 104, users can share personal tagged reference vital curves with their physician or primary care provider (PCP). A user may elect to anonymously share his or her vital signs data with trusted vital sign groups.

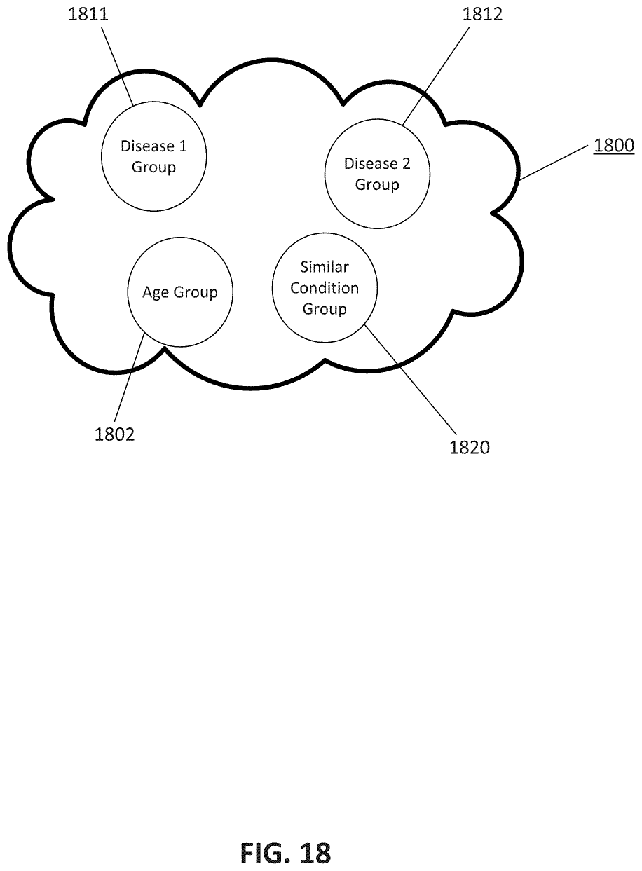

Referring momentarily to FIG. 18, each user of a global group 1800 in cloud storage provided by a server may elect to be part of a vital sign group (e.g., one or more of groups 1802, 1811,1812, 1820). Vital sign groups may be formed according to similar traits or similar conditions that the user has that may be shared anonymously by the user, such as age (e.g., group 1802), gender, height, weight, and/or infirmity. For example, a vital signs group (e.g., group 1820) might be composed of senior citizens with high blood pressure, diabetes and kidney conditions. Another vital signs group (e.g., group 1802) might be composed based on age, such as infants under the age of two for example. Another vital signs group (e.g., group 1811) might be composed based on a first disease, such as diabetes for example. Another vital signs group (e.g., group 1812) might be composed based on a second disease differing from the first disease, such as cancer for example. While the groups shown in FIG. 18 do not overlap, some groups may intersect sharing users between each group as the user may have both conditions of each group. A user having the characteristic disease or trait of a vital signs group can elect to be a member of some vital signs group or not. To be automatically included in the global group, a user operates the vital signs scanner 102 to obtain his/her vital signs, uses the multifunction device 104 to perform a login process to a group vital signs server agreeing to share his vital signs data, and uploads his/her vital signs data form the multifunction device 104 to the group vital signs server to share data. Further, to be automatically included in basic vital signs groups, such as age, gender, sex, height, and weight, a user has the underlying characteristic of the basic vital signs group, such as being a male for example of a male vital signs group.

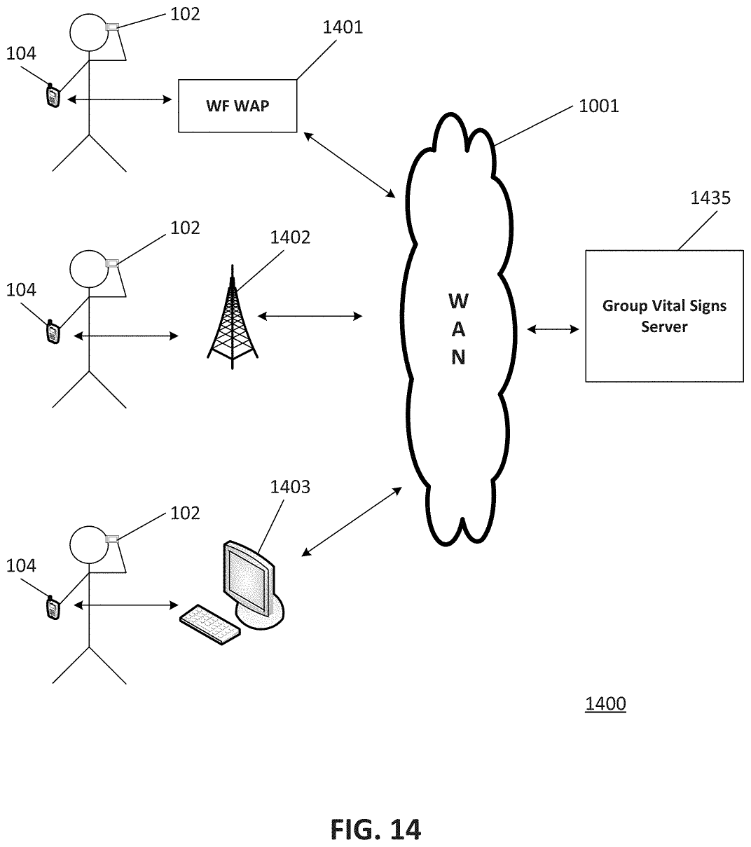

Referring now to FIG. 14, each user of the vital signs scanner 102 can elect to encrypt data and form a secure connection (e.g., an encrypted data connection) between a group vital signs server 1435 and their multifunction device 104 to exchange data over a network 1001.

The exemplary cloud server system 1400 depicted in FIG. 14 includes a plurality of vital sign scanners 102, and a plurality of multifunction devices 104 in communication with a group vital signs server 1435. The multifunction devices 104 can connect to the group vital signs server 1435 in different manners. Some multifunction devices 104 may connect to a network 1001 and the group vital signs server 1435 through a wireless access point (WAP) 1401. Other multifunction devices 104 may connect to the network 1001 and the group vital signs server 1435 through a wireless cellular data network 1402. Other multifunction devices 104 may make a wired connection with their personal computer 1403 that in turn is connected to the network 1001 and the group vital signs server 1435.

As with any cloud based application, the security of personal information can be important. Users can retain control of their group vital sign associations to maintain security of their personal information. Users can select which vital signs group they wish to belong and may change group affiliation as they see fit. The user may also select the people with whom they can share data. Only trusted people and primary care providers (PCPs) are given strict authorization to access the user's personal information. To others when sharing, the vital signs data is anonymous.

One exemplary method of maintaining information security is to limit the type of information that is transferred to the group vital signs server 1435. In one embodiment, only non-identifying (anonymous) information about the user is transferred to the group vital signs server. For example, each user can upload only their aggregated vital signs processed data.

The ability to select and change group affiliation also allows a user to compare their personal vital signs curves to different group reference vital signs curves. This feature can be beneficial if the user suspects that they are starting to develop a new chronic condition and wish to compare themselves against a different group before visiting their PCPs for an actual diagnosis. Potentially, the PCPs could recommend the user continue monitoring their vital signs at home using the vital signs scanner 102 and vital signs scanning software application 140 executed by the multifunction device 104 with follow-up office visits, saving both time and medical expense.

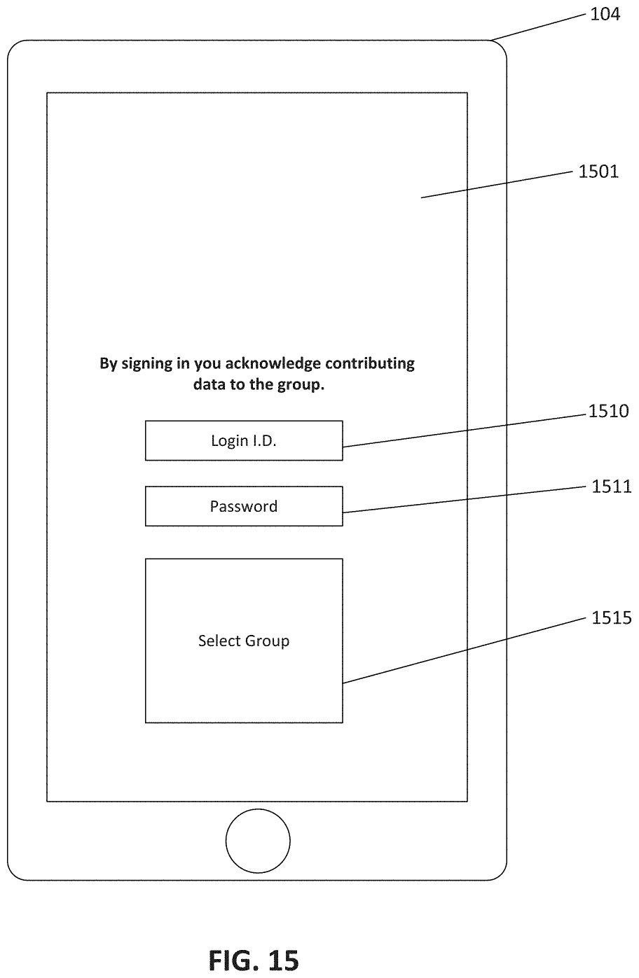

Referring now to FIG. 15, an exemplary user acknowledgment screen 1501 generated by the software application 140 is shown displayed by the display screen of the multifunction device 104. The screen 1501 includes a login warning statement “By signing in you acknowledge contributing data to the group.” The screen 1501 includes a login identification (ID.) entry field 1510 and a password entry field 1511 that the user completes to log into the group vital signs server 1435. By logging in, the user acknowledges that they are contributing data to the group. The screen 1501 further includes a vital signs group selection field or menu 1515.

The group vital signs server login process provides extra security and reduces the chance that personal data will be shared inappropriately with a wrong group. The user controls what information is shared with the selected vital sign groups and has to authenticate their identification before sharing is allowed. The user's device 104 can then upload his/her data to the group vital signs server 1435. With user vital sign data uploaded to the selected vital signs groups, the group vital signs server 1435 can cluster reference curves of single vital sign measurements of different users together to provide group reference vital sign curves.

Each member of a vital signs group performs scans at different times throughout the day. Thus, a group vital signs curve plotted strictly by the time of day may not accurately portray the group's average. The group vital signs server 1435 may optionally time warp the individual vital sign curves by performing clustering algorithms around standard events that most people perform throughout the day, such as eating breakfast, lunch, and dinner. Time warping shifts the vital signs data in time to make the given vital sign scan data more relevant.

Another feature of the group vital signs server 1435 is the aggregation and clustering of reference vital sign curves of multiple vital sign measurements. Mathematically, this means that the group vital signs server 1435 clusters multidimensional data together, rather than clustering individual measurements independently. The aggregation and clustering of reference vital sign curves can provide a more accurate description of the base map of a certain group of people. For example, senior citizens with high blood pressure, diabetes and kidney conditions may be anonymously grouped together for comparison with a user having the same conditions.

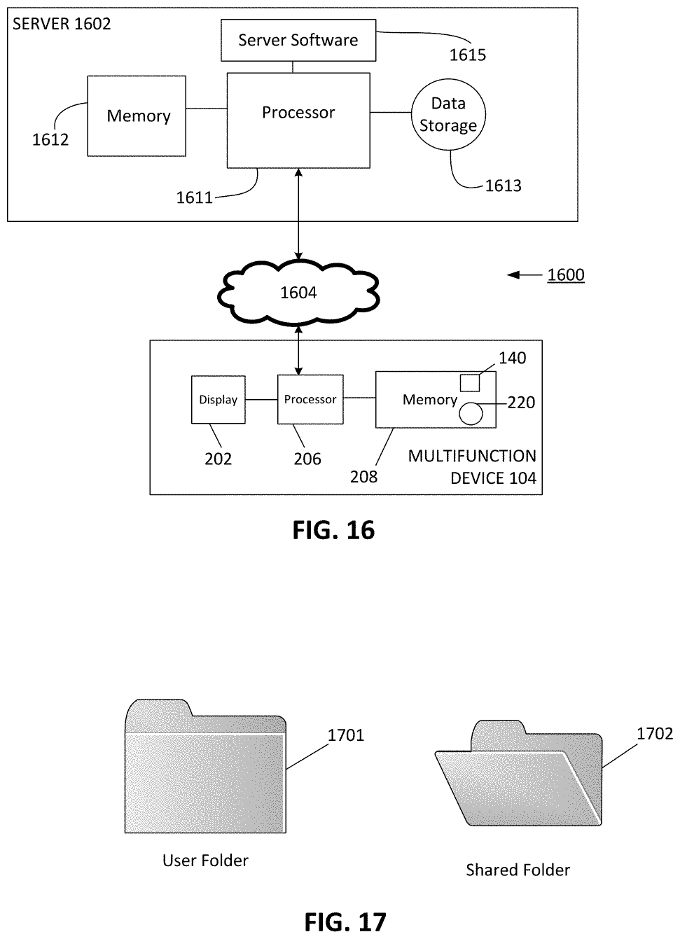

FIG. 16 is a functional diagram of a cloud based vital signs client server system 1600 in accordance with one embodiment of the invention. The vital signs client server system 1600 includes a server 1602 and one or more multifunction devices 104, the client, in communication with the server 1602 over an internet cloud 1604.

The server 1602 includes a processor 1611, a memory 1612, and one or more storage devices (e.g., hard disk drives) 1613 to store instructions of vital signs server software 1615 and vital signs data. The processor 1611 executes instructions of the vital signs server software 1615 to carry out a number of the server related functional processes described herein.

The portable multifunction device 104 includes a processor 206 and a non-volatile memory 208. The non-volatile memory 208 of the personal wireless digital device 104 may store the vital signs scanning software application 140 and the user data 220 related to the vital signs scanning software application. The processor 206 can read and write to the non-volatile memory 208 such that the vital signs scanning software application can provide a user interface to a user via the touch screen display device 202.

Processor 206 executes instructions of the vital signs scanning user interface (VSUI) software application 140. The VSUI software application 140 may include instructions for creating canonical vital signs curves, updating canonical vital signs curves with successive vital signs scan data, tagging vital signs scans, and periodically uploading vital signs data to the cloud and downloading group canonical curves.

Periodically, vital signs data may be transferred via the internet 1604 to the server 1602. Uploaded vital signs data may be stored in data storage devices 1613 and/or non-volatile server memory 1612. The vital signs server software 1615 may be executed by the processor 1611 to facilitate uploading and downloading of user vital signs data and group vital signs data.

FIG. 17 illustrates an exemplary method of organizing the user's data within a cloud system. Within the cloud data storage, a user may upload their data including personal data such as name, date of birth, address, infirmities, etc. into an encrypted user folder 1701. Within the cloud data storage may also be another folder 1702 that contains the user's shared vital signs data. All the data sent to the cloud data storage will be encrypted on the multifunctional device 104 before being sent to cloud. Only the user can decrypt their encrypted data using their specific multifunction device 104 or another device authorized by the user. The user can also authorize their PCPs to decrypt this data.

The data stored within the shared folder 1702 is medical grade data. This data is uploaded from the user's multifunction device 104 with full encryption and is not editable by the user. The user can choose and authorize whom they wish to share this data, for example, the user can share this data with their PCP and certain groups, but the user cannot edit this data. The user can also share their data with their groups, but preferably only non-identifying data would be shared with the group. For example, the group may receive the user's accumulated vital signs data along with the user's age, gender, and medical condition, but the user's name, address and other non-essential information would not be associated with this data. In either case, whether sharing with a PCP or with a group, the data is not editable by the user. A physician needs accurate unedited data to correctly diagnose the user and groups need accurate unedited data to form true vital sign group curves in order to provide all users with useful information.

FIG. 18 illustrates a diagram of a global group 1800 of users. The global group 1800 includes a plurality of vital sign groups 1802,18011,1812,1820 of a subset of users. A user may belong to one or more vital sign groups depending upon their condition. The user can choose to share their medical grade data with their groups. By belonging to a group, the user receives group canonical vital signs curve data.

Group canonical curves can be compared with a user's personal canonical vital signs curves. As illustrated in FIG. 11C, the multifunction device 104 can display on its display screen a chart of both a days vital signs curve 1103 and a group canonical curve 1113 for the same vital sign. A user can visually compare the curves to see the difference between them. As illustrated in FIG. 11D, the multifunction device 104 can display on its display screen a chart of both the user's canonical average curve 1106 and a group canonical curve 1116 for the same vital sign. The user again can visually compare the curves displayed on the display screen to see differences between them.

In some cases, the difference between a user's curve 1103,1106 and the group curve 1113,1116 may be beneficial, such as in the case of lower blood pressure for example. In other cases, the difference between curves may be detrimental, such as in the case of higher blood pressure for example. From differences between the user data and the group data, conclusions or observations may be automatically made by the vital signs software application 140 and shared with the user and/or his doctor or primary care provider.

The comparison of the user's personal canonical vital signs curves and the group canonical curves can be used by the user to better understand his/her basic health condition. The comparison of the user's personal canonical vital signs curves and the group canonical curves can be used by a doctor to help make a diagnosis of the user, if needed. Group vital signs information from the group vital signs server may be made valuable to doctors and public health professionals to monitor current trends in vital signs groups. The vital signs software application 140 may provide specific recommendations to the user (e.g., take more vital signs scans with the vital signs scanner 102 to gather additional vital signs data) based on the user's affiliation with one or more vital signs groups.

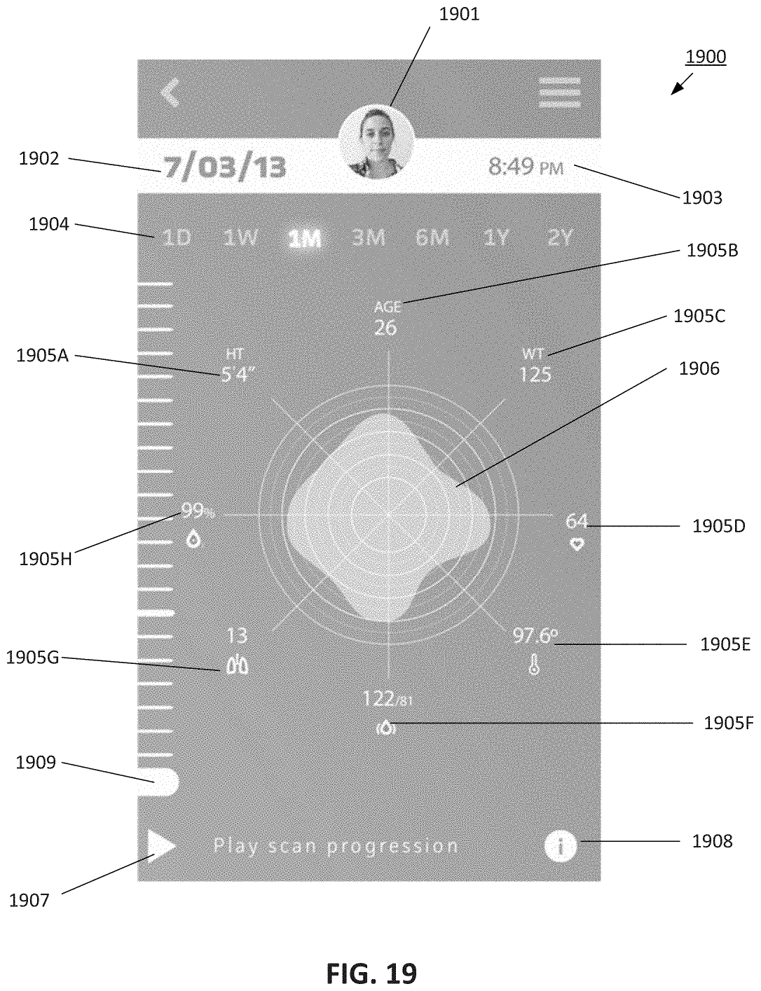

FIGS. 11A-11B illustrate static graphs for a single vital sign of a user. FIG. 19 illustrates an alternate way to display multiple vital signs of a user's vital sign history in an easy to read comprehensive manner by using a dynamic multidimensional graph.

Referring now to FIG. 19, a user interface 1900 is displayed on a display device of the multifunction device 104. The user interface 1900 includes an exemplary multidimensional graph 1906 of a user's vital signs. The user's vital signs are indicated along a plurality of axes or spokes radiating out from a center point with a curve being formed from values between each spoke. The multidimensional graph resembles a spider web and is often referred to as a spider graph 1906. The spider graph 1906 also allows the user to see their vital signs dynamically change over a selectable progression of time or history.

The user interface 1900 further includes a profile icon 1901 that may be the user's photo or some other icon or avatar chosen by the user. The profile icon 1901 identifies the user so that multiple users of a multifunction device 104 are aware of whose profile they are viewing. The user interface 1900 further includes a date display 1902 and a time display 1903 of the present date and time and may be provided by the multifunction device's calendar function.

The user interface 1900 further includes an interval bar 1904 that shows the user which progression interval is currently being displayed by the user interface. The progression interval of vital signs that may be displayed are for vital sign scans within a predetermined period of time such as one day (1D icon), one week (1W icon), one month (1M icon), three months (3M icon), six months (6M icon), one year (1Y icon), or two years (2Y icon) for example. The interval bar 1904 may allow selection of the progression interval by touching the icon for the predetermined interval. For example, the 1M icon may be touched in the touch sensitive display screen such that the progression interval is one month. The 1M icon is highlighted to indicate it is the progression interval that is desired to be displayed. In this case, the progression of vital signs displayed are within one month of the date 1902 as shown by the highlighted 1M icon.

Underneath the interval bar 1904, and forming axes of the spider graph 1906 are user characteristics and selected vital signs data.

The three user characteristics shown in the upper hemisphere of the graph 1906 are height 1905A, age 1905B, and weight 1905C that may be obtained by user input or by interfacing with other components, such as an electronic scale. If the multifunction device is equipped with a camera, another application may be able to photograph the user next to a linear chart and automatically derive the user's height by comparison to the linear scale. The user's age may be calculated from the present date 1902 and the user's date of birth input into the software application 140.

The other characteristics 1905D-1905H plotted along the other spokes or axes of the graph 1906 are vital signs obtained by the user through the use of the vital signs scanner 102. Each vital sign is shown as a number and an icon representing the vital sign measured. For example, 1905D shows a heart representing the user's heart rate and 1905E shows a thermometer representing the user's body temperature. The position of these characteristics around the cardinal axis can be rearranged based on the user's choice. The graph 1906 is an amalgam of the different user characteristics or vital signs 1905A-1905H.

The user interface 1900 may further include a slider bar 1909 that changes or selects a predetermined time of day about which information is desired. The user may slide the slider bar 1909 to select the measurement at a particular time of day that are desired for display. For example, a user may want to display measurements of vital signs data for vital sign scans that occurred around 8 P.M.

Play button 1907 activates the progression. When the progression is activated the software application 140 displays the change in the characteristics 1905A-1905H over the period of time selected. Certain characteristics just as age and height are likely to remain unchanged over the period of one month, but other vital signs may be more dynamic. The spider graph allows the user to visually see the change in their vital signs over a period of time. If the shape of the user's spider graph changes drastically, the user is more likely to be alerted to the change than if the results were displayed only numerically in a list.

A group vital sign curve 1950 of a plurality of vital signs associated with a vital signs group may also be displayed along the axes of the spider graph. The group vital sign curve 1950 selected by the user for comparison remains fixed as the user vital sign curve 1906 progresses over the selected period of time.

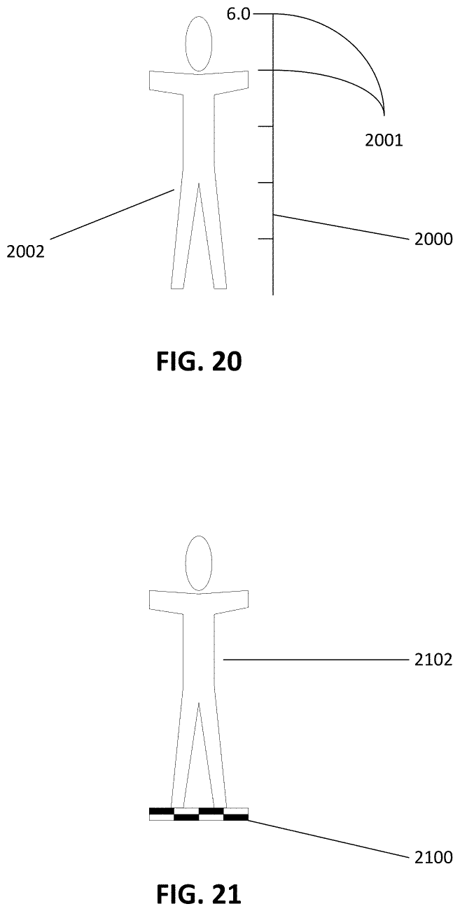

FIGS. 20 and 21 illustrate methods of automatically obtaining the user's height and weight, without needing manual input by the user. Regardless, the user's height and weight may be measured manually and manually input into the vital signs software application 140 and associated with the given date and time.

In FIG. 20, the user 2002 is illustrated standing next to a linear chart 2000. A photograph function on the user's multifunction device 140 can be used to photograph the user 2002 standing next to markings 2001 on the linear chart 2000. A third party application linked to the vital signs software application 140 or a sub-routine in the vital signs software application 140 may derive the user's height by comparison of the user's body position with the respect to the markings on the linear chart. Detecting the end point of the user's head against the marking on the linear chart 2000 indicates the height measurement of the user 2002. The height measurement is then reported to the vital signs software application 140 to be recorded as a characteristic of the user. The height measurement may change over time, particularly in younger users of the vital signs software application.

Referring to FIG. 21, a user's weight may be automatically measured and reported to the vital signs software application 140 as a user characteristic. A user 2102 stands on an electronic scale 2100 as shown in FIG. 21. The electronic scale 2100 is in communication with the multifunction device 104 and the vital signs scanning software 140. The electronic scale 2100 measures the user's weight each time the user 2102 steps on the scale 2100 and communicates each weight measurement to the vital signs software application 140. Each weight measurement for the user 2102 can be time stamped with date/time and recorded by the software application 140 for inclusion as a user characteristic and monitored vital sign. One can expect that a user's weight varies over the user's age and date/time and can generally have an influence on a user's health.

### Conclusion

When implemented in software, the elements of the embodiments of the invention are essentially the code segments or instructions to perform the functional tasks described herein. The code segments or instructions are executable by a processor, such as processor 206,840, and can be stored in a storage device or a processor readable storage medium, such as memory 208,841, awaiting execution. The processor readable storage medium may include any medium that can store information. Examples of the processor readable storage medium include an electronic circuit, a semiconductor memory device, a read only memory (ROM), a flash memory, an erasable programmable read only memory (EPROM), a floppy diskette, a CD-ROM, an optical disk, a hard disk. The code segments or instructions may be downloaded via computer networks such as the Internet, Intranet, etc. into the processor readable storage medium.

While certain embodiments of the disclosure have been described, these embodiments have been presented by way of example only, and are not intended to limit the scope of the disclosure. Indeed, the novel methods, systems, and devices described herein may be embodied in a variety of other forms. Furthermore, various omissions, substitutions and changes in the form of the methods, systems, and devices described herein may be made without departing from the spirit of the disclosure. For example, certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. Accordingly, the claimed invention is to be limited only by patented claims that follow below.

1-29. (canceled)

30. A method of tracking vital signs, the method comprising
performing a plurality of scans of a user's vital signs a plurality of times during each day over a plurality of days to obtain vital signs data;
aggregating the plurality of scans for each of the plurality of times over the plurality of days into a plurality of vital sign points associated with a plurality of joint time points; and
displaying the plurality of vital sign points along a canonical curve on a display device to inform a user of an average of a vital sign during each day over the plurality of days.

31. The method of claim 30, wherein
the plurality of points are displayed as ovals along the canonical curve to reflect standard deviations in the user's vital signs from the average value during each day.

32. The method of claim 30, further comprising:
adjusting a time of day associated with the vital signs data to a nearest joint time point to time warp the vital signs data together over the plurality of days.

33. A method of tracking vital signs, the method comprising
scanning a user's vital signs a plurality of times during a first day;
tagging a scan with user tag information; and
displaying the user's vital signs scan for the first day as a first curve.

34. A method of tracking vital signs, the method comprising
subscribing to a group or category or people;
scanning a user's vital signs a plurality of times during a plurality of days collect vital signs data;
encrypting the vital signs data;
sending vital signs data to a group server for inclusion into the group's vital signs data;
receiving average vital sign data of the group or category of people;
displaying the average vital sign data of the group or category of people as a group vital sign curve; and
displaying the user's vital sign curve alongside the group's vital sign curve.

35. The method of claim 34, further comprising:
prior to sending the user's vital signs data to the group server,
providing login information associated with the user's vital signs data to the group server;
sending a selection of one or more vital sign group associated with the user's vital signs data.
